(19) United States
(12) Patent Application Publication
(10) Pub. No.: US 2023/0285186 A1
(43) Pub. Date: Sep. 14, 2023

(54) COMPOSITIONS, SYSTEMS AND METHODS FOR TREATING BROWN FAT AND BEIGE FAT

(71) Applicant: MIRAKI INNOVATION THINK TANK LLC, Cambridge, MA (US)

(72) Inventors: Emilia JAVORSKY, Watertown, MA (US); Rainuka GUPTA, Newton, MA (US); Karen E. MILLER, South Dartmouth, MA (US); Tarik CHAUDHRY, Boston, MA (US)

(73) Assignee: MIRAKI INNOVATION THINK TANK LLC, Cambridge, MA (US)

(21) Appl. No.: 18/019,565

(22) PCT Filed: Aug. 6, 2021
(86) PCT No.: PCT/US21/44968

§ 371 Date: Feb. 3, 2023

Related U.S. Application Data
(60) Provisional application No. 63/062,680, filed on Aug. 7, 2020.

(51) Int. Cl. A61F 7/12

(52) U.S. Cl. CPC A61F 7/12; A61F 2007/126

(57) ABSTRACT

Compositions, systems and methods are provided for the treatment of subcutaneous brown or beige fat or visceral brown or beige fat, wherein the compositions comprise a cold solution comprising liquid water and/or ice particles, and optionally one or more additives and/or pharmacologic agents. The treatment of subcutaneous or visceral brown or beige fat provides activation of brown adipocytes, proliferation of brown adipocytes, increased volume of brown adipocytes, differentiation of precursors into beige and/or brown adipocytes, activation of beige adipocytes, stimulation of beige adipocytes and/or cryolipolysis of white adipocytes. The methods of treatment result in increased metabolic health, weight loss, and improved health.

### TECHNICAL FIELD

This disclosure is directed to compositions, systems and methods for treating brown fat and beige fat, wherein the fat is visceral or subcutaneous, in order to activate brown adipocytes, stimulate proliferation of brown adipocytes, increase volume of brown adipocytes, differentiate precursors into brown adipocytes, activate beige adipocytes, differentiate precursors into beige adipocytes, stimulate proliferation of beige adipocytes, and/or destroy white adipocytes.

### BACKGROUND

The human body comprises adipose tissue made of brown, beige and white adipocytes. Brown and beige adipocytes are thermogenic, i. e., they produce heat through metabolic stimulation. Brown adipocytes are able to disperse large amounts of chemical energy as heat through non-shivering thermogenesis, thus burning calories and potentially resulting in weight loss.

Brown adipocytes are present in many (possibly all) adults, and the amount thereof is inversely related to an increase in body mass index (BMI) or obesity. In adults, brown adipocytes are located between the shoulder blades, in the neck area, the inter-scapular region, the supraclavicular area, the mediastinum (para-aortic) area, the paravertebral area, the suprarenal area, the area surrounding the kidneys, and areas along the gastrointestinal tract. See FIG. 1. Beige and white adipocytes are located throughout the adipose tissue in the body. Beige adipocytes exist irregularly in white adipose tissue, and become prevalent through chronic exposure to cold, exercise, or treatment with particular pharmaceutical agents, such as a PPARγ agonist. Such a phenomenon is known as the “browning” of white adipose tissue, wherein the expression of uncoupling protein 1 (UCP-1) is increased.

White, beige and brown adipocytes may be found in both the subcutaneous adipose tissue and visceral adipose tissue. Subcutaneous adipose tissue (SAT, subcutaneous fat) is fat stored just beneath the skin, and is present in varying amounts that generally correlate with genetic and lifestyle factors. Subcutaneous fat helps store energy for the body, provides minor thermoregulation through insulation, and provides a layer of protection for muscles and bones against potential injury through impact. However, subcutaneous fat may impact health, fitness, and appearance, and has been shown to play a role in metabolic dysfunction and systemic inflammation in human subjects, and leads to serious health issues. It is difficult for many individuals to reduce subcutaneous fat through diet and exercise alone.

In some areas of the body, such as the torso, the subcutaneous adipose tissue is divided into two layers separated by a fascial plane. The upper layer is called the “superficial subcutaneous adipose tissue” (sSAT). The sSAT is characterized by a lamellar pattern having regular, defined cuboid fat lobules tightly organized within vertically oriented fibrous septae. The lower layer is called the “deep subcutaneous adipose tissue” (dSAT). The dSAT is characterized by a loose areolar pattern and has fat lobules are flat shaped, irregular in size, and are surrounded by high amounts of loose connective tissue. Both sSAT and dSAT layers also comprise sublayers.

Visceral fat is fat which is stored within the abdominal cavity, and located near the liver, pancreas, stomach and intestines. Subjects with a high amount of visceral fat also have increased health risks, including type 2 diabetes, insulins resistance, and heart disease.

Visceral brown fat may be found in the perivascular space around the aorta, common carotid artery, brachiocephalic artery, paracardial mediastinal fat, epicardial coronary artery and cardia veins, internal mammary artery, intercostal artery and vein; the periviscus space around the heart, trachea, major bronchi at lung hilum, esophagus, greater omentum, and transverse mesocolon; and around solid organs including thoracic paravertebral, pancreas, kidney, adrenal, liver and hilum of spleen. Subcutaneous brown fat may be found in between anterior next muscles and supraclavicular fossa, under the clavicles, in the axilla, in the interior abdominal wall, and in the inguinal fossa.

Methods available for treating (e.g., activating and/or increasing volume of) brown fat include topical or environmental cold based interventions, e.g., cold vest, cold room (air cooling), ice exposure on legs, β3 agonists such as mirabegron, GLP-1 agonists such as Pioglitazone or Sitagliptin, bariatric surgery, exercise, or supplements such as rutin, creatinine, capsacinoids, retinoids and 1-arginine.

Methods available for activating brown fat include topical cooling, e.g., with a cold pack, patch, vest, pillow, optionally in combination with pharmaceutical treatment, electrical stimulation, nerve stimulation, weight loss and surgical procedures; light activation, e.g., with infrared, UV or visible light; and internal cooling via implantable devices or refrigeration, optionally in combination with neurostimulation.

Engineered brown fat has been created using isolation and expansion of adipose stem cells and endothelial cells from white fat, induced by contact with certain differentiation factors to differentiate into brown fat and form 3D cell aggregates. The engineered brown fat is then injected or implanted into tissue.

However, there are many limitations to the available methods. Topical cooling treatments (such as those used in topical cryolipolysis) are lengthy, as the cold temperature needs to diffuse through the skin to the underlying subcutaneous fat. Further, topical cryolipolysis relies on an applicator which greatly limits the anatomic areas that can be treated (i.e., an area can only be treated if it can be accommodated by a standard applicator). Topical cryolipolysis also lacks precision, as the cold diffuses in an uncontrolled manner over a broad area during lengthy treatment times that are necessary for topical application. Because cooling of the fat can only be achieved by diffusion of cold through the skin to the subcutaneous fat, this greatly limits the depth and amount of fat that can be treated.

Conventional non-invasive and minimally-invasive fat removal methods, such as topical cooling and other energy-based therapies, are limited by depth, and fail to target brown fat. Accordingly, improved compositions, systems and methods for treatment of brown fat and beige fat are needed.

### LITERATURE

1. Sidossis et al., “Brown and beige fat in humans: thermogenic adipocytes that control energy and glycose homeostatis”, The Journal of Clinical Investigation. Col. 125, No. 2, February 2015, pp 478-486.
2. Abdullahi et al., “White Adipose Tissue Browning: A Double-edged Sword”, Trends in Endocrinology & Metabolism, Vol. 27, Issue 8, August 2016, pp 542-552.
3. U.S. Pat. No. 10,335,437.
4. Van der Lans et al., “Cold-activated brown adipose tissue in human adults: methodological issues”, Am J Physio Regul Integr Comp Physiol, 307, R1-3-R113, 2014.

### SUMMARY

The presence of brown and beige adipocytes in a human has beneficial effects, such as improved metabolic function, weight loss and improved health. Accordingly, there is a desire to provide methods for treating brown fat and beige fat effectively and efficiently, in order to activate brown fat cells, stimulate proliferation of brown fat cells, increase volume of brown fat cells, as well as induce differentiation into brown and beige fat cells, activate beige fat cells, stimulate proliferation of beige fat cells and/or destroy white fat cells.

The present invention provides compositions, systems and methods for treating brown fat and beige fat, by administering a cold solution to a subject in need thereof. The methods of the invention include selecting and targeting particular treatment site(s) in order to achieve the desired effects in the specifically desired location(s). Additionally, the methods of the invention allow quick, targeted administration of the cold solution, for example by injection, to the specifically chosen treatment site(s).

The present invention provides:
(1) A method for treating subcutaneous brown fat and/or subcutaneous beige fat, said method comprising administering an effective amount of a cold solution to a treatment site of a subject, wherein the treatment site is superficial subcutaneous adipose tissue, deep subcutaneous adipose tissue or superficial subcutaneous adipose tissue and deep subcutaneous adipose tissue, and wherein the cold solution comprises liquid water and/or solid ice particles.
(2) A method for treating visceral brown fat and/or visceral beige fat, said method comprising administering an effective amount of a cold solution to a treatment site of a subject, wherein the treatment site is visceral adipose tissue, and wherein the cold solution comprises liquid water and/or solid ice particles.
(3) The method for treatment according to the above (1), wherein the cold solution comprises about 2% to about 70% solid ice particles, and optionally one or more additives.
(4) The method for treatment according to the above (1), wherein the cold solution comprises about 71% to about 100% solid ice particles, and optionally one or more additives.
(5) The method for treatment according to the above (1), wherein the cold solution is substantially liquid, and optionally comprises one or more additives.
(6) The method for treatment according to the above (2), wherein the cold solution comprises about 2% to about 70% solid ice particles, and optionally one or more additives.
(7) The method for treatment according to the above (2), wherein the cold solution comprises about 71% to about 100% solid ice particles, and optionally one or more additives.
(8) The method for treatment according to the above (2), wherein the cold solution is substantially liquid, and optionally comprises one or more additives.
(9) A method for activating brown fat, said method comprising administering an effective amount of a cold solution to a treatment site of a subject in need thereof, wherein the treatment site is selected from the group consisting of superficial subcutaneous adipose tissue, deep subcutaneous adipose tissue, superficial subcutaneous adipose tissue and deep subcutaneous adipose tissue, visceral adipose tissue, visceral adipose tissue and superficial subcutaneous adipose tissue, visceral adipose tissue and deep subcutaneous adipose tissue, or visceral adipose tissue, superficial subcutaneous adipose tissue and deep subcutaneous adipose tissue, wherein the cold solution comprises liquid water and/or solid ice particles, and optionally one or more additives, and wherein the method results in activation of brown adipocytes, proliferation of brown adipocytes, increased volume of brown adipocytes, cryolipolysis of white adipocytes, conversion of white adipocytes into beige or brown adipocytes, conversion of white precursors into beige adipocytes, conversion of beige precursors into beige adipocytes, and/or conversion of brown precursors into brown adipocytes.
(10) The method for treatment according to the above (9), wherein the cold solution comprises about 2% to about 70% solid ice particles, and optionally one or more additives.
(11) The method for treatment according to the above (9), wherein the cold solution comprises about 71% to about 100% solid ice particles, and optionally one or more additives.
(12) The method for treatment according to the above (9), wherein the cold solution is substantially liquid, and optionally comprises one or more additives.
(13) The method for treatment according to any of the above (9) to (12), wherein prior to administering an effective amount of the cold solution, imaging the treatment site of the subject to determine the presence of brown adipocytes, beige adipocytes, white adipocytes, and/or precursors of brown, beige and/or white adipocytes.
(14) The method for treatment according to any of the above (9) to (13), wherein after administering an effective amount of the cold solution, imaging the treatment site, in order to monitor the activation of brown fat.
(15) The method for treatment according to any of the above (9) to (14), wherein the cold solution is administered in a single treatment, or in a series of treatments.
(16) The method for treatment according to any of the above (9) to (15), wherein the cold solution is administered via a device selected from the group consisting of a needle, an expandable needle, a needle comprising more than one tip, a fenestrated needle, a fenestrated cannula, and an implant.
(17) A method for increasing metabolic function, said method comprising administering an effective amount of a cold solution to a treatment site of a subject in need thereof, wherein the treatment site is selected from the group consisting of superficial subcutaneous adipose tissue, deep subcutaneous adipose tissue, superficial subcutaneous adipose tissue and deep subcutaneous adipose tissue, visceral adipose tissue, visceral adipose tissue and superficial subcutaneous adipose tissue, visceral adipose tissue and deep subcutaneous adipose tissue, or visceral adipose tissue, superficial subcutaneous adipose tissue and deep subcutaneous adipose tissue, wherein the cold solution comprises liquid water and/or solid ice particles, and optionally one or more additives, and wherein the method results in activation of brown adipocytes, proliferation of brown adipocytes, increased volume of brown adipocytes, cryolipolysis of white adipocytes, conversion of white adipocytes into beige or brown adipocytes, conversion of white precursors into beige adipocytes, conversion of beige precursors into beige adipocytes, and/or conversion of brown precursors into brown adipocytes, thus increasing the metabolic function of the subject.
(18) The method for treatment according to the above (17), wherein the cold solution comprises about 2% to about 70% solid ice particles, and optionally one or more additives.
(19) The method for treatment according to the above (17), wherein the cold solution comprises about 71% to about 100% solid ice particles, and optionally one or more additives.
(20) The method for treatment according to the above (17), wherein the cold solution is substantially liquid, and optionally comprises one or more additives.
(21) The method for treatment according to any of the above (17) to (20), wherein prior to administering an effective amount of a cold solution, imaging the treatment site of the subject to determine the presence of brown adipocytes, beige adipocytes, white adipocytes, and/or precursors of brown, beige and/or white adipocytes.
(22) The method for treatment according to any of the above (17) to (21), wherein after administering an effective amount of the cold solution, imaging the treatment site, in order to monitor the activation of brown fat.
(23) The method for treatment according to any of the above (17) to (22), wherein the cold solution is administered in a single treatment, or in a series of treatments.
(24) The method for treatment according to any of the above (17) to (23), wherein the cold solution is administered via a device selected from the group consisting of a needle, an expandable needle, a needle comprising more than one tip, a fenestrated needle, a fenestrated cannula, and an implant.
(25) A composition comprising a cold solution, wherein:
   the cold solution (1) comprises about 2% to about 70% solid ice particles, (2) comprises about 71% to about 100% solid ice particles, or (3) is substantially liquid;
   the composition optionally comprises at least one additive; and
   the composition is used to treat brown fat in a subject.
(26) A method for activating brown adipocytes in a subject in need thereof, said method comprising:
   topically cooling the skin of the subject, at or near a treatment site for activation of brown adipocytes,
   administering a β3 receptor agonist to the subject,
   imaging the treatment site and surrounding area,
   administering a cold solution to the treatment site of the subject via injection to activate brown adipocytes,
   wherein the first and second cold solution may be the same or different, and wherein the first and second cold solutions independently comprise liquid water and/or ice particles, and optionally one or more additives.
(27) A method for treating brown adipose tissue in a subject, said method comprising:
   imaging one or more potential treatment sites in the subject to determine the presence of brown adipocytes,
   selecting one or more treatment sites which contain brown adipocytes, and
   administering a cold solution into to the one or more selected treatment sites to treat the brown adipose tissue of the subject,
   wherein the cold solution comprises liquid water and/or ice particles, optionally one or more additives, and optionally one or more suitable pharmacological agents.
(28) A method for treating brown adipose tissue in a subject, said method comprising:
   imaging one or more potential treatment sites in the subject to visualize sympathetic innervation of adipose tissue, and
   administering a cold solution into a treatment site with sympathetic innervation,
   thereby treating brown adipose tissue,
   wherein the cold solution comprises liquid water and/or ice particles, optionally one or more additives, and optionally one or more suitable pharmacological agents.
(29) A method for treating brown and/or beige adipocytes in a subject, said method comprising:
   identifying treatment sites comprising brown and/or beige adipocytes by thermal imaging of the subject,
   selecting one or more treatment sites containing brown and/or beige adipocytes,
   administering a cold solution to the one or more selected treatment sites to treat the brown and/or beige adipocytes of the subject,
   wherein the cold solution comprises liquid water and/or ice particles, optionally one or more additives, and optionally one or more suitable pharmacological agents.
(30) A method for treating brown and/or beige adipocytes in a subject, said method comprising:
   selecting a treatment site known to contain brown and/or beige adipocytes based upon anatomical location,
   administering a cold solution to the one or more selected treatment sites of the subject to treat the brown and/or beige adipocytes of the subject,
   wherein the cold solution comprises liquid water and/or ice particles, optionally one or more additives, and optionally one or more suitable pharmacological agents.
(31) A method for forming beige adipocytes in a subject, said method comprising:
   imaging one or more treatment sites of the subject to determine the presence of white precursors,
   administering a cold solution to one or more treatment sites containing white precursors and the surrounding area in order to form beige adipocytes from the white precursors,
   wherein the cold solution comprises liquid water and/or ice particles, optionally one or more additives, and optionally one or more suitable pharmacological agents.
(32) A method for inducing cryolipolysis of white adipocytes and formation of beige adipocytes in a subject,
   imaging one or more treatment sites of the subject to determine the presence of white precursors,
   administering a cold solution to one or more treatment sites containing white precursors and the surrounding area in order to form beige adipocytes from the white precursors and to induce cryolipolysis of surrounding white adipocytes,
   wherein the cold solution comprises liquid water and/or ice particles, optionally one or more additives, and optionally one or more suitable pharmacological agents.
(33) A method for forming beige and/or brown adipocytes in a subject, said method comprising:
   imaging one or more treatment sites of the subject to determine the presence of beige and/or brown precursors,
   administering a cold solution to one or more treatment sites containing brown and/or beige precursors and the surrounding area in order to form beige and/or brown adipocytes from the beige and/or brown precursors,
   wherein the cold solution comprises liquid water and/or ice particles, optionally one or more additives, and optionally one or more suitable pharmacological agents.
(34) A method for forming beige and/or brown adipocytes in a subject and inducing cryolipolysis of white adipocytes, said method comprising:
   imaging one or more treatment sites of the subject to determine the presence of beige and/or brown precursors,
   administering a cold solution to one or more treatment sites containing brown and/or beige precursors and the surrounding area in order to form beige and/or brown adipocytes from the beige and/or brown precursors, and to induce cryolipolysis of surrounding white adipocytes,
   wherein the cold solution comprises liquid water and/or ice particles, optionally one or more additives, and optionally one or more suitable pharmacological agents.
(35) A method for inducing cryolipolysis of white adipocytes and effecting transdifferentiation from white adipocytes to beige adipocytes, said method comprising:
   imaging one or more treatment sites of the subject to determine the presence of beige adipocytes,
   administering a cold solution to white adipocytes surrounding the beige adipocytes, thus inducing cryolipolysis of a portion of the white adipocytes while leaving one or more white adipocytes remain, and to effect transdifferentiation from the remaining white adipocytes to beige adipocytes,
   wherein the cold solution comprises liquid water and/or ice particles, optionally one or more additives, and optionally one or more suitable pharmacological agents.
(36) A method for inducing cryolipolysis of white adipocytes and effecting transdifferentiation from white adipocytes to beige adipocytes, said method comprising:
   imaging one or more treatment sites of the subject to determine the presence of beige adipocytes,
   administering a first cold solution to white adipocytes surrounding the beige adipocytes, thus inducing cryolipolysis of a portion of the white adipocytes, while leaving one or more white adipocytes remain, and
   administering a second cold solution to effect transdifferentiation from the remaining white adipocytes to beige adipocytes,
   wherein the first and second cold solution may be the same or different, and wherein the first and second cold solutions independently comprise liquid water and/or ice particles, and optionally one or more additives.
(37) A system for treating brown adipose tissue, the system comprising:
   an imaging device,
   a delivery device for delivering a cold solution comprising liquid water and/or solid particles to a treatment site of a subject, and
   a cold solution supply source configured to supply the cold solution.
(38) A system for inducing cryolipolysis of white adipocytes and effecting transdifferentiation from white adipocytes to beige adipocytes, the system comprising:
   an imaging device,
   a first delivery device for delivering a first cold solution comprising liquid water and/or solid particles to a treatment site of a subject, and optionally a second cold solution comprising liquid water and/or solid particles to a treatment site of the subject,
   optionally a second delivery device for delivering a second cold solution comprising liquid water and/or solid particles to a second treatment site of the subject,
   wherein the first and second cold solution may be the same or different,
   a first cold solution supply source configured to supply the first cold solution and optionally the second cold solution, and
   optionally a second cold solution supply source configured to supply the second cold solution.
(39) A method for increasing the volume of brown fat in a subject, said method comprising imaging one or more treatment sites of the subject to determine the presence of brown adipocytes,
   administering a cold solution to the one or more treatment sites comprising brown adipocytes to activate and proliferate the brown adipocytes,
   and optionally imaging the one or more treatment sites to determine the increase in volume of brown adipocytes,
   wherein cold solution comprises liquid water and/or ice particles, and optionally one or more additives.
(40) A method for increasing the volume of brown fat in a subject, said method comprising selecting one or more treatment sites of the subject comprising brown adipocytes,
   wherein the one or more treatment sites are selected from the adipose tissue between the shoulder blades, in the neck region, in the inter-scapular region, in the supraclavicular area, in the mediastinum area, in the paravertebral area, in the suprarenal area, in the area surrounding the kidneys, and in areas along the gastrointestinal tract,
   imaging the one or more treatment sites to determine the amount of brown adipocytes present therein,
   administering a cold solution to the one or more treatment sites to activate and proliferate the brown adipocytes present therein,
   and optionally imaging the one or more treatment sites to determine the increase in volume of brown adipocytes,
   wherein cold solution comprises liquid water and/or ice particles, and optionally one or more additives.
(41) The method according to the above (40), wherein the cold solution is administered in a single treatment, or as a series of treatments.
(42) The method according to the above (40) or (41), wherein the cold solution is administered via a device selected form the group consisting of a needle, an expandable needle, a needle comprising more than one tip, a fenestrated needle, a fenestrated cannula, and an implant.
(43) The method according to any of the above (40) to (42), wherein the cold solution is administered in a single treatment, or as a series of treatments.
(44) The method according to any of the above (40) to (43), wherein the cold solution is administered via a device selected from the group consisting of a needle, an expandable needle, a needle comprising more than one tip, a fenestrated needle, a fenestrated cannula, and an implant.

### BRIEF DESCRIPTION OF THE DRAWINGS

FIG. 1 illustrates locations of brown adipose tissue in a human adult.

FIG. 2 provides lateral, anterior, and posterior view of the body, and indicates treatment sites which are suitable for the methods of treatment described herein.

FIG. 3 illustrates an example device for delivering a cold solution.

FIG. 4 illustrates an expandable needle configured to inject a cold solution.

FIG. 5 illustrates the expandable needle of FIG. 4, in an expanded state.

FIG. 6 illustrates a needle having a plurality of tips configured to impart a fractional cooling pattern.

FIG. 7 illustrates a needle having a plurality of radially extending of tips configured to impart a fractional cooling pattern.

FIG. 8 illustrates a non-limiting example of a fenestrated sheath, such as a fenestrated cannula or needle.

FIG. 9 illustrates a non-limiting example of a multi-lumen device.

FIG. 10 illustrates a method of treating brown fat.

### DETAILED DESCRIPTION

The invention provides compositions, systems and methods for treating brown fat and beige fat. The treatment of brown fat and beige fat includes treatment of subcutaneous fat or visceral fat, with a cold solution as defined herein, in order to activate brown adipocytes, expand cell populations of brown adipocytes, activate beige adipocytes, cause differentiation of precursors into desired beige and/or brown adipocytes, expand cell populations of beige adipocytes, and/or induce cryolipolysis of lipid-rich white adipocytes. Brown adipocytes are activated by cold temperatures, such as exposure of a subject to external cold temperatures, and thus are perfectly suited for activation by the methods of the present invention, which administers the cold solution defined herein directly to adipose tissue, fat cells, precursors thereof, and/or surrounding tissue. White adipocytes are removed by cold temperatures, such as exposure of a subject to external cold temperatures, and thus are perfectly suited for removal by the methods of the present invention, which administers the cold solution defined herein directly to adipose tissue, fat cells, precursors thereof, and/or surrounding tissue.

Activating brown adipocytes, activating beige adipocytes, increasing the volume of brown adipocytes, and/or increasing the volume of beige adipocytes improves the overall health of a subject, by increasing metabolism, regulating body weight, enhancing performance, controlling blood sugar, improving insulin levels, increasing longevity and providing anti-aging benefits.

The broad target areas for the methods of treatment include, but are not limited to, subcutaneous brown fat cells, visceral brown fat cells, subcutaneous white fat cells, visceral white fat cells, subcutaneous beige fat cells, visceral beige fat cells, white fat precursors, beige fat precursors, brown fat precursors, depots containing one or more of the targeted fat cells and/or precursors, and areas containing or surrounding one or more of the targeted fat cells and/or precursors. Precursors include pre-adipocytes, mesodermal stem cells and adipose tissue-precursor cells.

The specific target area or treatment site may be selected based upon known anatomical locations of brown, beige and/or white adipocytes and/or their precursors, or may be selected through imaging. When selecting a target area or treatment site through imaging, a known imaging device and/or method is utilized to evaluate or determine the presence of brown, beige and/or white adipocytes and/or their precursors. Imaging prior to treatment is also utilized to establish a baseline for comparison with later images to evaluate the progress and/or efficacy of the treatment method.

In one aspect, to activate brown adipocytes in a subject in need thereof, the target area may be between the shoulder blades, the neck, the inter-scapular region, the supraclavicular area, the mediastinum (para-aortic) area, the paravertebral area, the suprarenal area, the area surrounding the kidneys, and areas along the gastrointestinal tract, i.e., areas where brown adipocytes are present in adult humans, such that administration of the cold solution activates the brown adipocytes. In another aspect, to activate brown adipocytes, the target area may be selected by visualization through a known imaging method, such that specific locations of brown adipocytes may be identified and treated with the cold solution to activate the brown adipocytes.

In another aspect, to increase the volume of brown adipocytes in a subject in need thereof, the target area may be between the shoulder blades, the neck, the inter-scapular region, the supraclavicular area, the mediastinum (para-aortic) area, the paravertebral area, the suprarenal area, the area surrounding the kidneys, and areas along the gastrointestinal tract, i.e., areas where brown adipocyte precursors are present in adult humans, such that brown adipocyte precursors may be differentiated to form brown adipocytes by administration of the cold solution, thus increasing the volume of brown adipocytes. In another aspect, to increase the volume of brown adipocytes in a subject in need thereof, the target area may be adipose tissue throughout the body, such that brown adipocyte precursors may be differentiated to form brown adipocytes by administration of the cold solution, thus increasing the volume of brown adipocytes. In another aspect, to increase the volume of brown adipocytes in a subject in need thereof, the target area may be selected by visualization through a known imaging method, such that specific locations of brown adipocyte precursors may be selected, and caused to differentiate into brown adipocytes by administration of the cold solution, thus increasing the volume of brown adipocytes.

In some aspects, to increase the volume of brown fat in a subject in need thereof, the target area comprises known anatomical locations where brown adipocytes are present, as described above, or may be selected by visualization through a known imaging method, such that specific locations of brown adipocytes may be selected, wherein administration of the cold solution to the target area stimulates the brown adipocytes to proliferate, thereby increasing volume of brown fat. The increase in volume may be monitored visually through imaging, by measuring temperature changes in the target area and surrounding tissue, by measuring uptake of “free fatty acids” from disrupted white fat cells, by measuring increased perfusion to the area, and/or by measuring metabolic outcomes including but not limited to basal metabolic rate, fasting glucose and insulin tolerance test.

In some aspects, the method of treatment comprises activation and proliferation of brown fat by administration of a cold solution to a treatment site of a subject in need thereof. Such methods include identification of a target area by selection of one or more known anatomical locations of brown fat, and/or by visualization through a known imaging method, followed by administration of the cold solution to activate the existing brown fat and to increase the volume of brown fat in the subject, thus aiding in improved metabolism and fat reduction. In some aspects, the cold solution comprises 0% ice, and a large volume of the cold solution is administered slowly to the subject, resulting in brown fat activation, followed by growth of additional brown fat cells. In some aspects, the administration of the cold solution may be performed repeatedly, until the desired results are achieved. The more than one administrations of the cold solution may be performed in a single treatment session, or may be separated by one or more hours, one or more days, one or more weeks, one or more months, or any combination thereof.

In another aspect, to activate beige adipocytes in a subject in need thereof, the target area may be adipose tissue throughout the body, or the target area may be selected by visualization through a known imaging method such that specific locations containing beige adipocytes may be identified, wherein the target area is treated with the cold solution to activate the beige adipocytes. In another aspect, to increase the volume of beige adipocytes in a subject in need thereof, the target area may be adipose tissue throughout the body, or the target area may be selected by visualization through a known imaging method such that specific locations containing beige adipocytes may be identified, wherein the target area is treated with the cold solution to stimulate the beige adipocytes to proliferate, thereby increasing the volume of beige adipocytes.

In another aspect, to form white adipocytes and/or beige adipocytes in a subject in need thereof, the target area may comprise white adipose tissue, such that white adipocyte precursors may be differentiated into white adipocytes, or beige adipocytes may be formed through directed differentiation by administration of the cold solution. In another aspect, to form beige adipocytes, the target area may comprise adipose tissue throughout the body, or the target area may be selected by visualization through a known imaging method such that specific locations of beige adipocyte precursors may be identified, wherein the target area is treated with the cold solution such that the beige adipocyte precursors may be differentiated to form beige adipocytes.

In another aspect, to effect cryolipolysis of white adipocytes in a subject in need thereof, the target area is white adipose tissue, such that the administration of the cold solution to the target area results in cryolipolysis of white adipocytes, and may also result in activation of beige adipocytes within the white adipose tissue. In some aspects, the target area comprises brown fat, such that the brown adipocytes are activated, resulting in increased metabolism, body weight regulation, increased longevity and anti-aging. In some aspects, the target area comprises brown fat, such that the brown adipocytes are stimulated to proliferate, thereby increasing brown fat tissue volume.

The methods may comprise treatment of one or more of the target sites described above.

In some aspects, visualization through imaging may be utilized to select the target area and/or to increase the accuracy of target area selection. The imaging may be performed using one or more known methods, including but not limited to Magnetic Resonance Imaging (MRI), Computer Topography (CT), ultrasound, Position Emission Tomography (PET), including with 18F-fluorodeoxyglucose (FDG) or HED/¹¹C-meta-hydroxyephedrine, thermal imaging, 3D imaging, temporary topical markers, e.g., radiopaque markers, image guided injection of slurry, or image guided injection of harvested, treated fat cells. Visualization through imaging allows for targeted administration of the cold solution to particular areas, as well as monitoring throughout treatment to determine the effectiveness of the treatment and/or to determine whether a new target area should be treated.

Uncoupling protein 1 (UCP1) is found in brown adipose tissue, and may be used to monitor the presence of, or activation of, brown and beige adipocytes. Specifically, when activated by administration of the cold solution, beige and brown adipocytes express UCP1, and therefore, monitoring the expression of UCP1 may be used to assess the metabolic activity of the tissue at or near the treatment site, and determine the efficacy of the methods of treatment by administration of the cold solution.

Clinical areas of interest for increasing the volume of brown and/or beige fat, and/or activating brown and/or beige fat include, but are not limited to, weight loss, enhancing performance, enhancing metabolism, treating obesity, treating metabolic syndrome, treating nonalcoholic fatty liver disease, treating inflammation, increasing lifespan and improving health span.

An obese person may contain many white adipocytes and few beige and/or brown adipocytes, resulting in decreased energy expenditure, increased body weight, decreased insulin sensitivity, and increased hepatic steatosis. A lean person may contain fewer white adipocytes and more beige and/or brown adipocytes than an obese person, thus resulting in increased energy expenditure, decreased body weight, and increased insulin sensitivity.

The methods of treatment according to the invention comprise administering a cold solution to a subject by any suitable method. The method of treatment will vary based upon the end result desired by the patient, and the patient's characteristics. The cold solution may comprise liquid water and/or ice particles. For example, the cold solution can be substantially liquid, substantially solid, or a slurry comprising both liquid and solid ice particles.

In some aspects, the cold solution may include water. In some aspects, the cold solution may include water and one or more additives. In some aspects, the one or more additives are inactive, biocompatible ingredients, including any substance (at or below their respective concentrations) in the FDA GRAS list, which is incorporated by reference in its entirety herein. In some aspects, the additives comprise one or more of a salt, a sugar, and a thickener.

In some aspects, the cold solution comprises potassium chloride at about 0.02% by mass or lower, for example, 0.19, 0.18, 0.17, 0.16, 0.15, 0.14, 0.13, 0.12, 0.11, 0.10, 0.09, 0.08, 0.07, 0.06, 0.05, 0.04, 0.03, 0.02, 0.01, or 0% by mass. In some aspects, the cold solution comprises calcium chloride at about 0.02% by mass or lower, for example, 0.19, 0.18, 0.17, 0.16, 0.15, 0.14, 0.13, 0.12, 0.11, 0.10, 0.09, 0.08, 0.07, 0.06, 0.05, 0.04, 0.03, 0.02, 0.01, or 0% by mass. In some aspects, the cold solution comprises sodium chloride at about 2.25% by mass or lower, for example at about 2.2, 2.1, 2.0, 1.9, 1.8, 1.7, 1.6, 1.5, 1.4, 1.3, 1.2, 1.1, 1.0, 0.9, 0.8, 0.7, 0.6, 0.5, 0.4, 0.3, 0.2, 0.1, 0.05 or 0% by mass. In some aspects, the cold solution comprises magnesium chloride at about 0.02% by mass or lower, for example, 0.19, 0.18, 0.17, 0.16, 0.15, 0.14, 0.13, 0.12, 0.11, 0.10, 0.09, 0.08, 0.07, 0.06, 0.05, 0.04, 0.03, 0.02, 0.01, or 0% by mass.

In some aspects, the cold solution comprises sucrose at about 5% by mass or lower, for example at about 5, 4.5, 4, 3.5, 3, 2.5, 2, 1.5, 1, 0.5, or 0% by mass. In some aspects, the cold solution comprises dextrose at about 5.6% by mass or lower, for example at about 5.5, 5, 4.5, 4, 3.5, 3, 2.5, 2, 1.5, 1, 0.5, or 0% by mass. In some aspects, the cold solution comprises mannitol at about 4.95% by mass or lower, for example at about 4.5, 4, 3.5, 3, 2.5, 2, 1.5, 1, 0.5, or 0% by mass. In some aspects, the cold solution comprises lactose at about 0.45% by mass or lower, for example at about 0.4, 0.35, 0.3, 0.25, 0.2, 0.15, 0.1, 0.05, or 0% by mass. In some aspects, the cold solution comprises sorbitol at about 4.7% by mass or lower, for example at about 4.5, 4, 3.5, 3, 2.5, 2, 1.5, 1, 0.5, or 0% by mass. In some aspects, the cold solution comprises glycerol at about 2% by mass or lower, for example at about 1.9, 1.8, 1,7, 1.6, 1.5, 1.4, 1.3, 1.2, 1.1, 1.0, 0.9, 0.8, 0.7, 0.6, 0.5, 0.4, 0.3, 0.2, 0.1, 0.05 or 0% by mass.

In some aspects, the cold solution comprises hetastarch at about 6% by mass or lower, for example at about 5.5, 5, 4.5, 4, 3.5, 3, 2.5, 2, 1.5, 1, 0.5, or 0% by mass. In some aspects, the cold solution comprises pectin at about 16.7% by mass or lower, for example at about 16, 15, 10, 9, 8, 7, 6, 5, 4, 3, 2, 1 or 0% by mass. In some aspects, the cold solution comprises polyethylene glycol at about 20% by mass or lower, for example at about 20, 15, 10, 9, 8, 7, 6, 5, 4, 3, 2, 1 or 0% by mass. In some aspects, the cold solution comprises gelatin at about 16% by mass or lower, for example at about 15, 10, 9, 8, 7, 6, 5, 4, 3, 2, 1 or 0% by mass. In some aspects, the cold solution comprises sodium methylcellulose at about 5% by mass or lower, for example at about 5, 4.5, 4, 3.5, 3, 2.5, 2, 1.5, 1, 0.5, or 0% by mass. In some aspects, the cold solution comprises a sodium alginate at about 5% by mass or lower, for example at about 5, 4.5, 4, 3.5, 3, 2.5, 2, 1.5, 1, 0.5, or 0% by mass. In some aspects, the cold solution comprises polyvinyl alcohol at about 5% by mass or lower, for example at about 5, 4.5, 4, 3.5, 3, 2.5, 2, 1.5, 1, 0.5, or 0% by mass. In some aspects, the cold solution comprises polyvinyl pyrrolidone (PVP) at about 5% by mass or lower, for example at about 5, 4.5, 4, 3.5, 3, 2.5, 2, 1.5, 1, 0.5, or 0% by mass. In some aspects, the cold solution comprises Xanthan Gum at about 0.75% by mass or lower, for example at about 0.7, 0.6, 0.5, 0.4, 0.3, 0.2, 0.1, 0.05 or 0% by mass. In some aspects, the cold solution comprises CMC at about 0.75% by mass or lower, for example at about 0.7, 0.6, 0.5, 0.4, 0.3, 0.2, 0.1, 0.05 or 0% by mass. In some aspects, the cold solution comprises guar gum at about 1% by mass or lower, for example at about 0.9, 0.8, 0.7, 0.6, 0.5, 0.4, 0.3, 0.2, 0.1, 0.05 or 0% by mass. In some aspects, the cold solution comprises locust bean gum at about 1% by mass or lower, for example at about 0.9, 0.8, 0.7, 0.6, 0.5, 0.4, 0.3, 0.2, 0.1, 0.05 or 0% by mass. In some aspects, the cold solution comprises gum tragacanth at about 1% by mass or lower, for example at about 0.9, 0.8, 0.7, 0.6, 0.5, 0.4, 0.3, 0.2, 0.1, 0.05 or 0% by mass. In some aspects, the cold solution comprises carbomer at about 1% by mass or lower, for example at about 0.9, 0.8, 0.7, 0.6, 0.5, 0.4, 0.3, 0.2, 0.1, 0.05 or 0% by mass.

Additional exemplary additives include bulking agents, such as sucrose, lactose, trehalose, mannitol, sorbitol, glucose, raffinose, glycine, histidine, PVP (K40); salts such as potassium, calcium, magnesium, hydrogen phosphate, hydrogen carbonate; buffering agents, such as sodium citrate, sodium phosphate, sodium hydroxide, tris base-65, tris acetate, tris HCl-65; tonicity modifiers, such as dextrose; collapse temperature modifiers such as dextran, ficoll, gelatin, and hydroxyethyl starch; antimicrobial preservatives such as benzalkonium chloride, benzethonium chloride, benzyl alcohol, chlorobutanol, m-cresol, myristyl gamma-picolinium chloride, paraben methyl, paraben propyl, phenol, 2-phenoxyethanol, phenyl mercuric nitrate, and thimerosal; chelating agents such as calcium disodium EDTA (ethylenediaminetetra acetic acid), disodium EDTA, calcium versetamide Na, calteridol, and DTPA; antioxidant and reducing agents such as acetone sodium bisulfate, argon, ascorbyl palmitate, ascorbate (sodium/acid), bisulfite sodium, butylated hydroxyl anisole, butylated hydroxyl toluene (BHT), 19roprano/cysteinateHCl, dithionite sodium, gentistic acid, gentistic acid ethanolamine, glutamate monosodium, glutathione, formaldehyde sulfoxylate sodium, metabisulfite potassium, metabisulfite sodium, methionine, monothioglycerol(thioglycerol), nitrogen, propyl gallate, sulfite sodium, tocopherol alpha, alpha tocopherol hydrogen succinate, thioglycolate sodium, thiourea, and anhydrous stannous chloride; solvents and co-solvents such as benzyl benzoate, oils, castor oil, cottonseed oil, N,N dimethylacetamide, ethanol, dehydrated ethanol, glycerin/glycerol, N-methyl-2-pyrrolidone, peanut oil, PEG, PEG 300, PEG 400, PEG 600, PEG 3350, PEG 4000, poppyseed oil, propylene glycol, safflower oil, sesame oil, soybean oil, vegetable oil, oleic acid, polyoxyethylene castor, sodium acetate-anhydrous, sodium carbonate-anhydrous, triethanolamine, and deoxycholate; buffers and pH-adjusting agents such as acetate, ammonium sulfate, ammonium hydroxide, arginine, aspartic acid, benzene sulfonic acid, benzoate sodium/acid, bicarbonate-sodium, boric acid/sodium, carbonate/sodium, carbon dioxide, citrate, diethanolamine, glucono delta lactone, glycine/glycine HCl, histidine/histidine HCl, hydrochloric acid, hydrobromic acid, lysine (L), maleic acid, meglumine, methanesulfonic acid, monoethanolamine, phosphate (acid, monobasic potassium, dibasic potassium, monobasic sodium, dibasic sodium and tribasic sodium), sodium hydroxide, succinate sodium/disodium, sulfuric acid, tartarate sodium/acid, and tromethamine (Tris); stabilizers such as aminoethyl sulfonic acid, asepsis sodium bicarbonate, L-cysteine, dietholamine, diethylenetriaminepentacetic acid, ferric chloride, albumin, hydrolyzed gelatin, 19roprano, and D,L-methionine; surfactants such as polyoxyethylene sorbitan monooleate (TWEEN® 80), Sorbitan monooleate, polyoxyethylene sorbitan monolaurate (TWEEN® 20), lecithin, polyoxyethylene-polyoxypropylene copolymers (PLURONICS®), polyoxyethylene monolaurate, phosphatidylcholines, glyceryl fatty acid esters, urea; complexing/dispersing agents such as cyclodextrins (e.g., hydroxypropyl-β-cyclodextrin, sulfobutylether-Bcyclodextrin); viscosity building agents such as celluloses such as sodium carboxymethylcellulose (CMC), hydroxyethylcellulose, hydroxypropylmethylcellulose, methylcellulose), acacia, gelatin, methyl cellulose, xanthan gum, polyethylene glycol, guar gum, locust bean gum, carrageenan, alginic acid, gelatin, carbopol, polyvinyl and pyrrolidone. Additives can be any of those found in Sougata Pramanick et al., “Excipient Selection in Parenteral Formulation Development,” 45(3) Pharma Times 65-77 (2013), which is incorporated herein by reference in its entirety.

In some aspects, the cold solution can include one or more therapeutic agents, for example, an antioxidant, an anesthetic, a vasoconstrictor, an antibacterial, and a neuroprotectant.

The cold solution can be delivered to a subject such as a human or an animal, therefore the solution can be sterile and have an osmolality and pH such that it does not harm target or non-target tissue. In some aspects, the cold solution may have an osmolality of less than about 2,200 milli-Osmoles/kilogram. In some aspects, the cold solution may have an osmolality of less than about 1,000 milli-Osmoles/kilogram. In some aspects, the osmolality may be less than about 600 milli-Osmoles/kilogram. In some aspects, the pH is between about 4.5 and about 9.

In some aspects, the cold solution is substantially liquid such as the cold solution described in PCT/US2019/55605 filed on Oct. 10, 2019, which is incorporated by reference in its entirety herein. The cold solution can be cooled or supercooled to a temperature just before spontaneous nucleation occurs. Alternatively, the cold solution can be cooled or supercooled to a temperature approximate to or lower than where spontaneous nucleation occurs, then warmed such that all ice particles melt prior to delivery to a subject. One example of a cold solution is water that is supercooled. Water normally freezes at 273.15 K (0° C. or 32° F.), but it can be supercooled at standard pressure down to its crystal homogeneous nucleation at almost 224.8 K (−48.3° C./−55° F.). The supercooling process requires that water be pure and free of nucleation sites. This can be done by processes like reverse osmosis or chemical demineralization. Rapidly cooling of water at a rate on the order of 10\hat{ }6 K/s avoids crystal nucleation and water becomes a glass, i.e., an amorphous (non-crystalline) solid. The temperature of the cold solution can be cooled to a temperature ranging from at about 10° C. to at about −50° C. One or more additives can be selected and included in the cold solution to change the freezing point of the cold solution.

In some aspects, the cold solution is substantially solid, i.e., substantially ice, such as the substantially solid solution. Substantially solid solutions, systems and methods for generating substantially solid solutions, and methods for administering substantially solid solutions are described in U.S. Provisional patent application Ser. No. 62/953,272 filed on Dec. 24, 2019, which is incorporated by reference in its entirety herein. For example, the cold solution can comprise ice at a concentration of about 71% to about 100%, including 71, 75, 80, 85, 90, 91, 92, 93, 94, 95, 96, 97, 98, 99, 99.5, 99.9 and 100%. In some aspects, the cold solution may comprise 95 to 100% of ice in a solid state, e.g., an ice composition. In some aspects, the cold solution comprising 95% to 100% ice in a solid state is an ice needle composition, which may be generated and/or delivered via a cannula such as a needle.

In some aspects, the cold solution is a slurry, including a liquid and solid ice particles, such as the slurry described in International Patent Applications PCT/US2019/54828 filed on Oct. 4, 2019 and PCT/US2015/047301 filed on Aug. 27, 2015, both of which are incorporated by reference in their entirety herein. Systems and methods for making a slurry are described in International Patent Application PCT/US2019/55634 filed on Oct. 10, 2019, which is incorporated by reference in its entirety herein. One or more additives can be selected to optimize flowability which is the ability of the slurry to flow through a device or within a subject. For example, flowability describes how easy it is for the slurry to move, either within the system for making the slurry, a delivery device for delivering the slurry such as a cannula, or within the body of a human subject. Flowability is dependent on several factors, including ice particle size, ice particle shape (as they relate to the configuration of the delivery device, for example, needle gauge) and viscosity.

The slurry includes ice particles, for example at a concentration of about 2% to about 70%. In some aspects, the ice concentration is about 20% to about 50%, for example, at about 30% to about 40%, for example, about 31, 32, 33, 34, 35, 36, 37, 38, or 39%. The ice particles can be substantially rounded and uniform in shape and size. Ice crystal size can be based upon the size of the delivery device, for example, an ice particle size of about 100 μm may allow injection through a needle having an inside diameter of about 1.0 mm or smaller. In some aspects, the ice particle size may be less than about 1 mm, or less than about 0.25 mm. In some aspects, the temperature of the slurry can range from about −25° C. to about 10° C., for example, from about −6° C. to about 0° C., for example, about −5° C., −4° C., −3° C., −2° C., −1° C.

The type of cold solution(s) (i.e., substantially liquid, substantially solid, or slurry) and characteristics thereof (e.g., osmolality, volume, temperature, ice content, ice shape/size) can be selected based on the characteristics of the treatment site and desired outcome. A single treatment can include the delivery of one or more types of cold solution to one or more treatment sites via any suitable delivery method and any combination thereof.

The cold solution may comprise one or more pharmacologic agents. In some aspects of the invention, the pharmacological agent includes, but is not limited to PPAR agonists, including but not limited to PPARγ-agonists such as thiazolidinediones, (e.g., pioglitazone, rosiglitazone, lobeglitazone), NSAIDs (e.g., ibuprofen, salicylates, propionic acid derivatives, acetic acid derivatives, enolic acid derivatives, selective COX-2 inhibitors), indole, fibrate drugs (e.g., clofibrate, gemifibrozil, ciprofibrate, bezafibrate), aleglitazar, muraglitazar, tesaglitazar, saroglitazar, caffeine, genistein, isoproterenol, theophylline, cysteine, gallic acid, rutin and catechin.

In some aspects of the invention, the pharmacological agent includes exogenous agonists, including but not limited to BDNF, catecholamines (e.g., catechol, dopamine, norepinephrine, epinephrine, fenofibrate), IL-6, PTHrP, Meteorin-like (METRNL), Irisin, Prostaglandins, VEGF, ANP/BNP, GDF5, FGF (FGF19, FGF21), BMPs (BMP4, BMP7, BMP8b). One or more exogeneous agonists may be incorporated into the cold solution.

In some aspects of the invention, the pharmacological agent includes drug agonists to promote endogenous signaling, including but not limited to BDNF, including but not limited to SSRI's (e.g., citalopram, escitalopram, fluoxetine, 22ropranolol, paroxetine, sertraline, dapoxetine) and SNRI's (e.g., atomoxetine, desvenlafaxine, duloxetine, levomilnacipram, milnacipran, sibutramine, tramadol, venlafaxine); catecholamines (e.g., amitriptyline, imipramine, nortriptyline, phenoxybenzamine, doxazosin, terazosin, prazosin, atenolol, metroprolol, 22ropranolol, labetolol, nifedipine, amlopdipine, diltiazem, verapamil, hydrazaline, isosorbide, minoxidil, ephedrine, pseudoephedrine, amphetamine, albuterol, caffeine, nicotine, theophyilline, levodopa, carbidopa); IL-6, including but not limited to antidepressants (e.g., venlafaxine, imipramine, serotonin, luoxetine); PTHrP, including but not limited to teriparatide, abaloparatide; Meteorin-like (METRNL); Irisin; prostaglandin analogues (e.g., travaprost, latanoprost, tafluprost, latanoprostene bunod, bimatoprost); VEGF (e.g., HIF1-alpha, PHD1, PHD2, PHD3, Azelnidipine, azilsartan, lercanidipine, nafcillin; ANP/BNP, including but not limited to sympathomimetics (methoxamine, midodrine, metaraminol, phenylephrine, amidephrine, clonidine, Dexmedetomidine, Fadolmidine, Guanfacine, Guanethidine, Xylazine, Tizanidine, Medetomidine, Methyldopa, Methylnorepinephrine, Norepin ephrine, Lofexidine, Medetomidine, Xylometazoline, Oxymetazoline, Cirazoline, Epinephrine, e rgotamine, etilefrine, indanidine, mephentermine, metaraminol, methoxamine, mivazerol, naphaz oline, norfenefrine, octopamine, phenylpropanolamine, propylhexedrine, rilmenidine, romifidine, synephrine, talipexole); GDF5; FGF (e.g., FGF19, FGF21, e.g., SIRT1 activators polyphenols such as reservatrol, methylene blue, metformin, NAD+); BMPs (e.g., BMP4, BMP7, BMP8b), including but not limited to apigein, disometin, isoliquirtigenin, and 4′-hydroxychalcone.

In some aspects of the invention, the pharmacological agent includes drug agonists, including but not limited to TGF-B, TNF-alpha and Retinoid Acid. In some aspects, the pharmacological agent includes β3 adrenergic receptor agonists, including but not limited to mirabegron, nebivolol and solabegron.

In some aspects of the invention, the pharmacological agent includes, but it not limited to, berberine, omega-3 fatty acids including alpha-linolenic acid, eicosapentaenoic acid, and docashexaenoic acid, melatonin, green tea extract, (−)-epigallocatechin-3-gallate (EGCG), menthol, ginsenoside, curcumin, artepillin C, bitter melon seed oil, butein, luteolin, farnesol, cryptotanshinone, albiflorin, trans-anethole, magnolol, xanthohumol, L-rhamnose, grape pomace extract, phytol, nobiletin, medicarpin, olaparib, dietary sea buckhorn pomace, zeaxanthin, trans-cinnamic acid, 6-gingerol, and apple polyphenols.

In some aspects of the invention, the pharmacological agent includes creatine.

In some aspects, more than one pharmacological agent may be administered. In some aspects, the one or more pharmacological agents may be administered prior to, concurrently with, or subsequent to administration of the cold solution. When the one or more pharmacological agents are administered concurrently with the administration of the cold solution, the one or more pharmacological agents may be administered independently from the cold solution, or may be present in the cold solution.

In some aspects, the one or more pharmacological agents may be orally administered to the subject as a supplement to the administration of the cold solution. A subject may be treated by administration of the cold solution to treat brown fat and/or beige fat, resulting in the activation and recruitment of brown and/or beige fat, followed by administration of an oral supplement comprising one or more pharmacological agents, in order to maintain the treatment results.

In some aspects, the cold solution may be administered in combination with engineered brown adipose tissue, e.g., as described in U.S. Pat. No. 10,335,437, the contents of which is incorporated herein by reference in its entirety.

The cold solution may be administered in a single treatment, or may be administered as a series of treatments, for example a pre-treatment, followed by a treatment, followed by a post-treatment. In some aspects, the pre-treatment, treatment and post-treatment may occur in the same session. In other aspects, the pre-treatment and post-treatment may occur in sessions before and after the treatment session, respectively. In some aspects, the pre-treatment may occur 24 hours prior to the treatment. In another aspects, the pre-treatment may occur 24 hours after the treatment. In some aspects, there may be multiple pre-treatments, multiple treatments, and/or multiple post-treatments, which may occur in a single treatment session, or may be separated by one or more hours, one or more days, one or more weeks, or one or more months.

The cold solution may be administered continuously, such as through a catheter, providing consecutive or simultaneous effects, including activation of existing brown adipocytes, and change from brown, beige and/or white precursors to metabolically active brown and/or beige adipocytes. In another aspect, the cold solution may be administered periodically, in order to provide one or more of the effects noted above. In some aspects, imaging may be provided between periodic administrations in order to determine the effectiveness of the treatment, to determine the treatment site and/or to determine whether a new treatment site should be selected.

The cold solution may be administered by administration into subcutaneous fat and/or visceral fat. For administration into subcutaneous and visceral fat, the cold solution may be injected into subcutaneous fat followed by injection into visceral fat, injection into visceral fat followed by injection into subcutaneous fat, or injection into visceral fat and subcutaneous fat simultaneously.

For administration to subcutaneous fat, the cold solution may be injected into sSAT only, injection into dSAT only, injection into dSAT followed by injection into sSAT, injection into sSAT followed by injection into dSAT, and injection into dSAT and sSAT simultaneously, thus allowing for selective targeting of sSAT and dSAT. In some aspects, injection into the dSAT followed by injection into the sSAT is utilized to allow for visualization of each layer during the injection.

Multiple treatments may be performed, for example with a first session targeting visceral fat and a second session targeting subcutaneous fat; or a first session targeting subcutaneous fat and a second session targeting visceral fat; or a first session targeting visceral fat, a second session targeting the sSAT, and a third session targeting the dSAT; or a first session targeting the sSAT, the second session targeting the dSAT, and a third session targeting the visceral fat, or any other iteration thereof. Any layer(s), e.g., visceral fat, sSAT and dSAT, can be treated in any order in any number of treatments. Multiple treatments of each session may be required, prior to, or intermixed with multiple treatments of other sessions. The multiple treatments may occur in the same treatment session, or may be spaced apart in hours, days, weeks or months.

To reduce pain associated with injection, methods of the invention may further comprise administering an anesthetic to an area for treatment of the subject prior to injecting the cold solution, topically and/or via injection. For example, the anesthetic may be a local anesthetic, such as lidocaine. In certain aspects, the anesthetic may be administered to a subject a suitable amount of time in advance of the treatment in order to numb the injection area before treatment of the cold solution. In certain aspects, the cold solution may be topically applied to numb the injection site prior to injection.

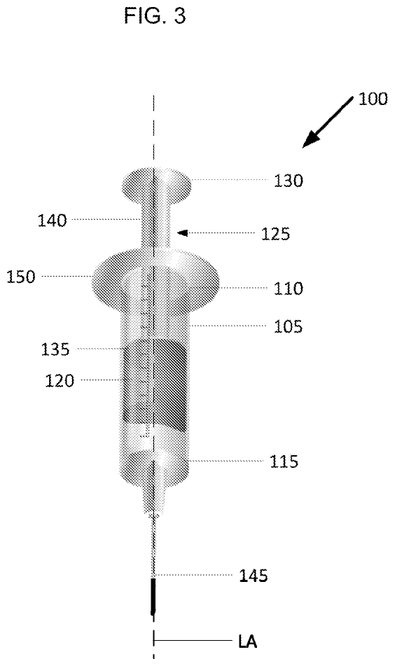

The cold solution is administered to a human subject by any suitable method. In some aspects, the cold solution is injected by any suitable means, such as injection by a delivery device, or a cannula such as a needle. An exemplary delivery device is generally shown in FIG. 3. The delivery device 100 includes a cylindrical member 105 having a first end 110 and a second end 115 along a longitudinal axis LA. The delivery device also includes an interior lumen 120 defined by the interior wall of the cylindrical member 105 and provided to receive and hold cold solution. The cylindrical member also includes a ledge 150, or flange, extending around the first end 110 out from the cylindrical member 105 along a plane that is orthogonal to the longitudinal axis LA. The ledge 150 also has an opening concentric with the interior lumen 120.

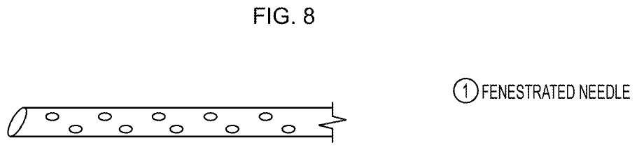

The ledge helps facilitate handling and delivery of cold solution from the delivery device 100. In some aspects, the delivery device 100 is a syringe-type device, for example, any suitable sterile syringe. The syringe can include a gauge size ranging from 8-25G. In some aspects, the delivery device comprises a fenestrated needle, for example, as shown in FIG. 8.

The cylindrical member 105 can be made of any type of biocompatible pharmacologically inert material suitable for use in holding and supplying fluids to be provided within a human body. Exemplary materials for the cylindrical member 105 include plastic, such as polyethylene or polypropylene, and glass. The delivery device 100 can be any size that suitable to hold one or more aliquots (doses) of cold solution for delivery to the desired tissue. As an example, the volume capacity of the delivery device 100 can be between 1 ml and 60 ml, although capacity outside of those volumes is also contemplated.

The delivery device 100 also includes a plunger 125 at least partially disposed within the interior lumen 120. The plunger 125 is configured to move in and out of the cylindrical member 105 through the first end 110. The plunger 125 includes a head 130, a plunging member 135, and a rod 140 extending between the head 130 and plunging member 135 along the longitudinal axis LA. The plunging member 135 is disposed along the rod 140 at a predetermined distance from the head 130. The delivery device 100 also includes at least one needle 145 extending from the second end 115. The needle 145 can comprise a gauge between 8 gauge and 25 gauge and a length between ¼ inch and 10 inches, such as about ¼ inch, ½ inch, 1 inch, 2 inches, 3 inches, 4 inches, 5 inches, 6 inches, 7 inches, 8 inches, 9 inches, or 10 inches. In some aspects, the cylindrical member 105 narrows or tapers to a small opening at the second end 115, where the small opening is configured to receive the needle 145. Preferably, the needle 145 is a hypodermic needle. Exemplary needle materials include, but are not limited to, stainless steel and carbon steel, with or without nickel platin.

The plunger 125, including the head 130 and the rod 140, can be any type of biocompatible, pharmacologically inert material suitable for coming in contact with fluids to be provided within a human body. Exemplary materials for the plunger 125 include plastic, such as polyethylene or polypropylene, and glass. With respect to the plunging member, a portion or all of the plunging member 135 can be a rubber material, such that a seal is formed between the sides of the plunging member 135 and the interior wall of the cylindrical member 105. The rubber material can be any rubber suitable for coming in contact with fluids to be provided to the human body, such as natural rubber latex or a synthetic rubber. In some aspects, the delivery device 100 can also include an agitator (not shown) disposed within the interior lumen 120 configured to mix the cold solution ingredients.

Once the cold solution is ready for delivery to tissue using the delivery device 100, the needle 145 is used to pierce the skin. Once the needle 145 is through the skin and positioned at or near the target tissue, i.e., treatment site, the plunger 125 is forced downward toward the second end 115 of the cylindrical member 105. The force of the plunging member 135 on the cold solution forces the cold solution through the cylindrical member 105, out the needle 145, and into (or near) the treatment site. In one embodiment, more than one needle is provided at the second end 115 of the delivery device 100. The more than one needle can be provided in single row array, multiple row array, circular pattern, or any other conceivable arrangement.

The needle may be any suitable type of surgical needle. In some aspects, the needle is a fenestrated needle, an example of which is shown in FIG. 8. The needle may be a surgical needle of any suitable size. In some aspects, the needle comprises a gauge size of about 8G to about 25G. In some aspects, more than one needle may be used for administration. The more than one needle can be provided in a single row array, multiple row array, circular pattern, or any other conceivable arrangement. Administration via a needle or needle array be may performed in conjunction with imaging, to enhance accuracy and avoid damage or puncture to surrounding areas. The needle or needle array may have a fixed-depth, such that it provides sufficient depth to administer the cold solution to the target area, while preventing damage or puncture to surrounding tissue and/or organs.

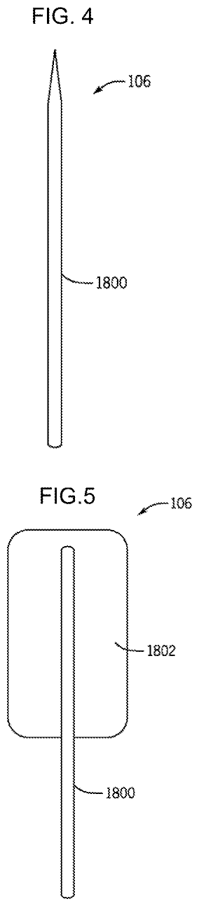

In some non-limiting examples, as shown in FIGS. 4 and 5, the delivery device 106 may comprise an expandable needle 1800. The expandable needle 1800 may be cooled by a cooling device and subsequently advanced by a user of the delivery device to a target area. As shown in FIG. 5, once the expandable needle 1800 reaches the desired target area, the user can expand a balloon 1802 attached to the expandable needle 1800. A cold solution at a desired temperature may then be delivered through the expandable needle 1800 to the balloon 1802 to provide cooling to the desired tissue region. It should be appreciated that the balloon 1802 may not need to be inflated prior to injection of the cold solution. Rather, injection of the cold solution may inflate the balloon 1802. Once the desired cooling treatment has been applied to the desired tissue region, the balloon 1802 may be retracted to in deflated state.

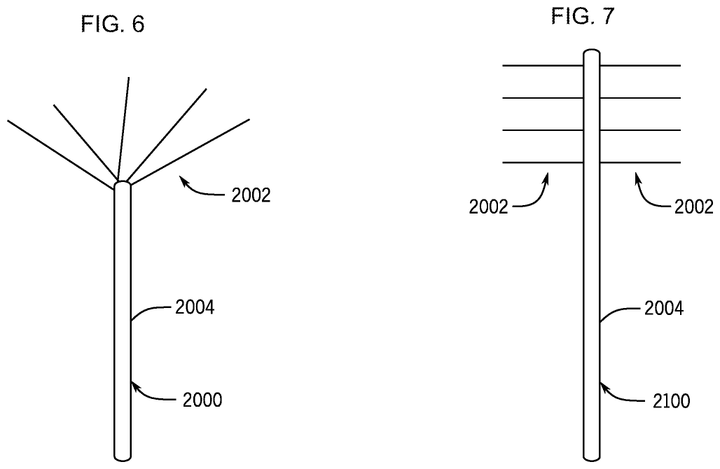

FIGS. 6 and 7 illustrate two non-limiting examples of fractional delivery arrays 2000 and 2100, which may be implemented in a delivery device. The fractional delivery array 2000 may be advanced by a user to a desired target area. Once the fractional delivery array 2000 is advanced to the desired target area, a cold solution may be delivered to the desired target area in a fractional pattern through a plurality of needles 2002. The plurality of needles 2002 can extend outwardly from a distal end of an array tube 2004. As shown in FIGS. 6 and 7, the plurality of needles 2002 may be arranged in alternative patterns to define alternative fractional cooling patterns, as desired.

In some aspects, the cold solution may be administered by an implantable device, including but not limited to a balloon or a custom 3D-printed hollow implant. The balloon or hollow implant may be filled with the appropriate cold solution for the specific treatment and/or specific target site. The balloon or hollow implant may be re-filled one or more times for continuous treatment. The refilling of the balloon or hollow implant may occur on a pre-determined schedule, or may occur on an as-needed basis, such as after evaluation of prior treatments through monitoring and/or imaging. The cold solution to be administered via an implantable balloon or custom 3D-printed hollow implant may comprise one or more pharmacological agents, in order to augment the effects of the cold solution. The hollow implant may be in the shape of a tube, permitting inflow and outflow to allow circulation of the cold solution in and around the implant.

In some aspects, multiple hollow implants may be 3D-printed, then filled with the desired cold solution, to be administered over multiple treatment cycles. All of the implants may be filled at the start of the treatment, or each implant may be filled prior to implantation. Such a method may include printing multiple 3-D printed hollow implants, filling the hollow implant to be implanted with the desired cold solution, monitoring the treatment site for increase in brown and/or beige fat cells, removing the implant when the amount of brown and/or beige fat cells stabilizes, filling a second 3D-printed hollow implant with the desired cold solution, which may be the same or different from the previously implanted cold solution, monitoring the treatment site for increase in volume of brown and/or beige fat cells, and removing the implant when the amount of brown and/or beige fat cells stabilizes. This method may be continued for multiple cycles, until the desired effect is achieved.

In some aspects, a cold solution, such as a substantially solid solution, can be administered to a treatment site, for example, via one or more incisions. In some aspects, the substantially solid solution can be generated using a mold where the mold can be 3D printed to represent the size and shape of brown fat at the treatment site(s). In some aspects, a substantially solid solution can be administered in combination with an electrical current. For example, a substantially solid solution can be formed around the exterior of an electrical probe where the substantially solid solution and electrical current can be used to stimulate innervation to brown or beige fat to increase activation of brown fat.

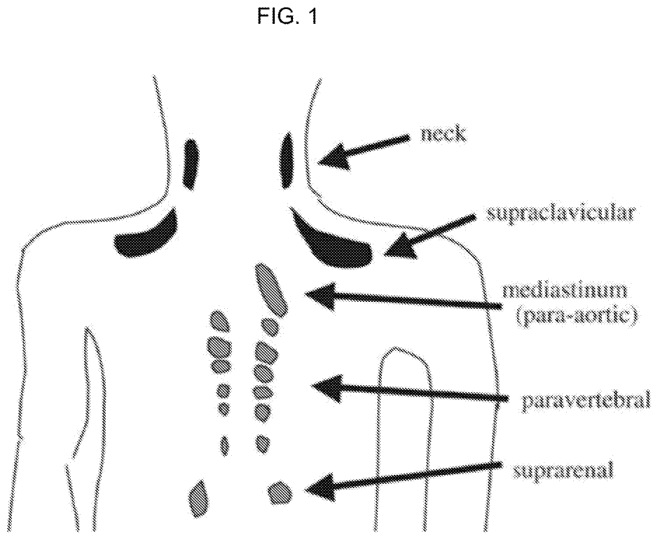

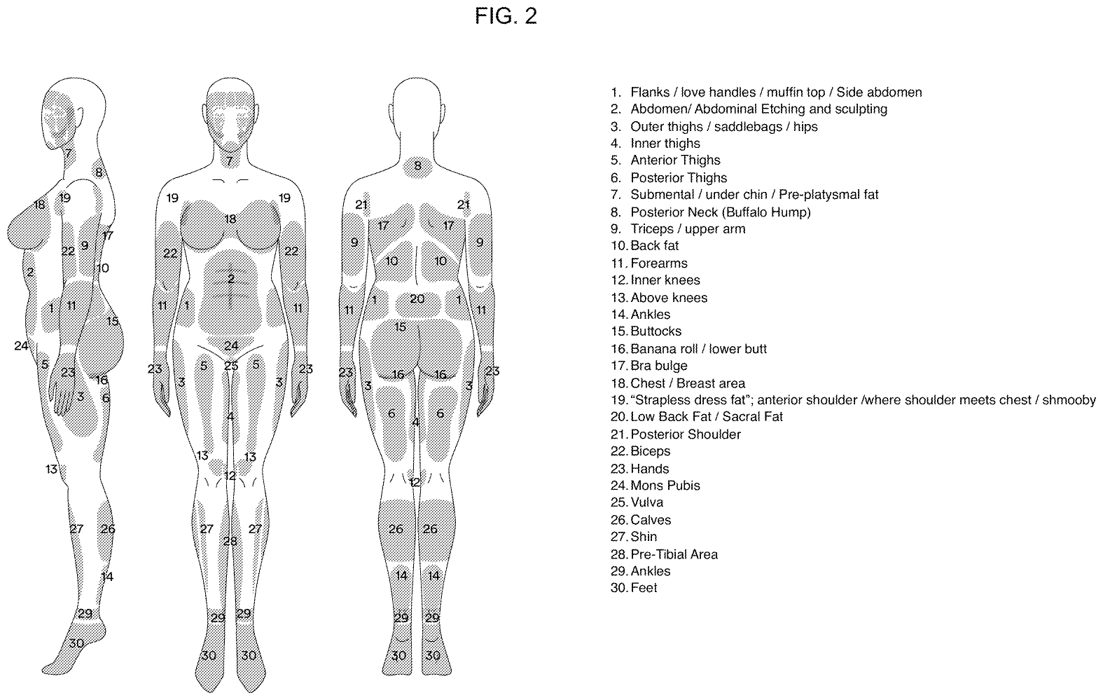

Any suitable amount of cold solution that is safe for administering to the subject may be injected, based on subject characteristics, the treatment site and/or to produce desired effects of treatment. Potential treatment sites are visually demonstrated in FIG. 2, which provides lateral, anterior and posterior images of the human body, and FIG. 1 which shows known locations of brown adipose tissue. A treatment may include delivering a volume of cold solution to one or more treatment sites. For example, when a cold solution is delivered via injection, the site may be treated via one or more injection sites, i.e., puncture site, and one or more deposition sites. The deposition site is where the cold solution is deposited, regardless of the injection site, and may be a different site than the injection site or the same site. One or more treatments can be required to achieve a desired effect.

In some aspects, where the treatment site is a larger area such as the abdomen and the delivery comprises injection, the amount of cold solution injected may be about 2 L or less per injection site. In some examples, the amount of cold solution injected is about 1 mL to about 2 L per injection site, for example, 1 mL, 2 mL, 3 mL, 4 mL, 5 mL, 6 mL, 7 mL, 8 mL, 9 mL, 10 mL, 11 mL, 12 mL, 13 mL, 14 mL, 15 mL, 16 mL, 17 mL, 18 mL, 19 mL, 20 mL, 25 mL, 50 mL, 75 mL, 100 mL, 125 mL, 150 mL, 175 mL, 200 mL, 225 mL, 250 mL, 275 mL, 300 mL, 325 mL, 350 mL, 375 mL, 400 mL, 425 mL, 450 mL, 475 mL, 500 mL, 525 mL, 550 mL, 575 mL, 600 mL, 625 mL, 650 mL, 675 mL, 700 mL, 725 mL, 750 mL, 775 mL, 800 mL, 825 mL, 850 mL, 875 mL, 900 mL, 925 mL, 950 mL, 975 mL, 1 L, 1.25 L, 1.5 L, 1.75 L and 2 L. For example, different subjects have different amounts of subcutaneous fat, and therefore, some subjects may require injection of greater amounts of cold solution in order to produce visible effects of reduction and removal of subcutaneous fat. Other subjects may require multiple treatments to produce the desired effects of treating brown fat.

Imaging may be utilized before, during, and/or after each treatment to form a baseline, monitor progress, and/or determine the efficacy of the treatment of brown fat. Additional measurements may be taken before, during and/or after each treatment, in order to determine the volume and/or activity of brown adipose tissue, weight loss, body fat mass, basal metabolic rate (kcal/day/kg fat-free body mass), insulin sensitivity (glucose tolerance test), hepatic lipid content (MR), VO2 Max/Stress Test, and/or to obtain a brown fat sample for research during anterior cervical spine surgery, parathyroidectomy or thyroidectomy.

In some aspects, where the treatment site is a smaller site such as soft tissue in the neck, the amount of cold solution injected may be about 1 mL to about 1 L per injection site. For example, a cold solution can be delivered to the entire outer circumference of the neck or any portion or portions thereof.

A cold solution for use in the invention may be injected at multiple treatment sites. In some aspects, the selected treatment sites may be visceral fat, the superficial subcutaneous fat layer, the deep subcutaneous fat layer, or any combination thereof. For example, the cold solution may be injected into the visceral fat, the superficial subcutaneous fat layer and/or the deep subcutaneous fat layer at a plurality of injection sites. In some aspects, the cold solution is injected at a plurality of injection sites into both subcutaneous fat layers, optionally in combination with injection at a plurality of injection sites in the visceral fat.

In some aspects, the superficial subcutaneous fat layer is treated at the same time as the deep subcutaneous fat layer. For example, the cold solution is injected into superficial subcutaneous fat, and then the needle is moved deeper in the deep subcutaneous fat regions. In an aspect, a fenestrated needle having a suitable length with fenestrations in both the first subcutaneous fat layer and the second subcutaneous fat layer is used to treat the first and second subcutaneous fat layers at the same time. In another aspect, the superficial subcutaneous fat layer and the deep subcutaneous fat layer are treated at the same time by injecting a needle and slowly withdrawing the needle, releasing cold solution in both subcutaneous fat layers.

In some aspects, the injection sites may form a pattern, such as a plow, fan, or grid-like pattern, or in a single bolus or multiple bolus injections. In some aspects, one injection site is used repeatedly, thereby reducing the number of injection sites and concomitant scarring potential. In a plow injection pattern, a single initial target injection site is used followed by a moving needle for additional deposition sites, for example in a linear pattern. In a fan injection pattern, deposition sites form an arc from 1 to 360 degrees. In a bolus injection, the cold solution is deposited in a single injection site.

The injection pattern and/or cold solution (including type and/or ingredients) can be determined based on the subject's profile, treatment plan (as described below), or based on the target site to be treated, i.e., treatment site. For example, an injection pattern and/or volume may be selected to optimize consistency of temperature at the treatment site. In an embodiment, the injection pattern and/or volume is selected in order to achieve gradient cooling of the tissue proximate to or at a treatment site or injection site. Injection techniques, including the patterns described herein, are known to those of skill in the art.

Treatment with the cold solution comprises reduction or removal of white adipocytes in the subcutaneous fat layers and/or in visceral fat in the human subject by freezing, or cryolipolysis. Treatment also comprises activation of brown adipocytes, proliferation of brown adipocytes, utilization of white, beige and/or brown adipocyte precursors, and/or activation and proliferation of beige adipocytes. Treatment may further comprise tightening of skin of the human subject. The tightening of the skin results from a collagen response upon removal and reduction of the fat cells in the subcutaneous fat layer. Reduction of subcutaneous fat may also reduce adipose tissue hypoxia or inflammatory signaling in overweight and obese individuals. Additionally, the cold solution may be utilized to mechanically disrupt fibrous tissue to break up compartments found within the subcutaneous fat, allowing the subcutaneous fat to spread and create a visually smoother appearance, for example in the treatment of cellulite.

Treatment with the cold solution may be optimized for cosmetic or aesthetic results, for example to achieve smoothing and to avoid the appearance of sharp edges in the subcutaneous layer or layers. In some aspects, a profile can be created that correlates to the ice coefficient in the cold solution. The ice coefficient is defined as the percentage of ice, i.e., the percent by volume of water in a solid state in the cold solution, or the amount of ice by weight. For example, a cold solution with a higher ice coefficient can be used to treat the center of a treatment site, while a cold solution with a lower ice coefficient can be used to treat to the outer perimeter of the treatment site. Any of the cold solution properties such as ice coefficient, ice size and ice shape, can be varied to achieve a desired result.

In an aspect of the invention, a treatment plan may be created for a subject, for example to determine one or more of type of cold solution, the cold solution properties (for example, ingredients, tonicity and/or ice content, amount of cold solution to be delivered, delivery method(s) (e.g., ingestion, inhalation, injection, topical/contact, and/or incision), treatment sites such as visceral fat, superficial and/or deep layers, target areas such as areas containing white adipocytes, brown adipocytes, beige adipocytes, and/or white, brown and/or beige precursors, a suitable device for administration of the cold solution, and whether to include one or more additional modalities in the subject's treatment. Factors considered in creating a treatment plan for a subject may comprise one or more of gender, height, body weight, body fat percentage, percentage of brown adipocytes, anatomy such as septae rigidity, lifestyle, vitals, medical history, lipid profiles, skin elasticity, medication, nutrition, supplements, demographic, vascularity of fat tissue, fat saturation, and the like. Fat saturation may be characterized by one or more of imaging, biopsy, and impedance measurement. In some aspects, after a plan is created for the subject, the amount of cold solution to be administered can be adjusted based on one or more of the area or areas to be treated, the areas to be treated, the depth of injection, and the injection pattern to be used, and which, if any, additional modalities are to be incorporated.

Imaging may also be utilized during creation of a treatment plan for a subject by collecting pre-, peri-, and/or post-injection data from one or more subjects. Information may be obtained through any suitable procedure, including but not limited to Magnetic Resonance Imaging (MRI), Computed Topography (CT), ultrasound, Positron Emission Tomography (PET), thermal imaging, Optical Coherence Tomography (OCT), and combinations thereof. Utilizing images of the target area, treatment site, surrounding areas, and/or other areas of interest, may provide detailed information regarding the most suitable treatment plan.

A computer or artificial intelligence system may be utilized to create a treatment plan and/or a post-procedure plan for a subject by collecting pre-, peri-, and/or post-injection data from multiple subjects. It is appreciated that the more data points, the more effective the artificial intelligence system will be in creating a treatment plan for a subject. For example, pre-, peri-, and/or post-injection data may be collected for each subject comprising one or more of gender, height, body weight, body fat percentage, the subject's anatomy, lifestyle, the subject's vitals, medical history, lipid profiles, skin elasticity, medication, nutrition, supplements, demographic, fat saturation, imaging data, treatment data and fat loss data. Data may be measured by any suitable means. For example, fat loss data may be measured by calipers or any imaging methods such as ultrasound, MM, 3D photography, visual assessment, and the like. In some aspects, the system can be used to determine treatment eligibility, to order pre-treatment diagnostics/health screening, provide cost estimates, simulate results, provide information for subjects about the procedure, assist with patient intake, provide connectivity to service providers and Telehealth options, and provide an option for a subject to share individual treatment goals with a provider.

In some aspects, pre-procedure monitoring, e.g., to assist in creation of a treatment plan, and/or post-procedure monitoring, and/or a treatment modality can be performed via one or more monitoring devices including but not limited to a wearable physiological monitoring device, a sleep monitoring device, a metabolic monitoring device, a glucose monitoring device, monitoring of various biomarkers associated with health/disease such as markers associated with inflammatory and oxidative stress, blood work monitoring, hormone monitoring, body waste monitoring, white to brown fat conversion rate monitoring, mental well-being monitoring, taking physical measurements, e.g., using measuring tape, muscle mass measurement, 3D Image Scan, bioelectric impedance measuring, e.g., a scale, a handheld device, whole body measuring device, a direct segmental whole body composition measuring device, hydrostatic weighing centers, measurement of rate of healing of torn muscle fibers from exercise, indirect calorimetry, and a wearable to passively measure oxygen intake and carbon dioxide output, e.g., an RBC measurement, e.g., a device worn on a nostril.

Pre- or post- treatment steps may be utilized to optimize treatment results. For example, a massaging step may be utilized to increase fat cell damage and/or the mechanical force of the ice in the cold solution. In an aspect, the massaging is performed to puncture one or more cell membranes. The massaging step may be used to position or shape the cold solution post injection. Massaging can be performed by any mechanical means, for example by hand, vibration, an applicator, or by acoustic means. Imaging pre-injection can be utilized to create a treatment plan and may further be used to develop the profile for the subject. For example, the septae of the subject may be damaged prior to injection of the cold solution to allow the cold solution to flow more smoothly. In an embodiment, the septae in damaged by puncture. In another aspect, the septae is damaged by massaging.

Prior to, during, or after administration of the cold solution to the treatment site, visualization and identification of the target tissue may be performed. In some aspects, the target tissue is one or more of subcutaneous brown fat, deep brown fat, subcutaneous white fat, deep white fat, subcutaneous beige fat, deep beige fat, white precursors, beige precursors or brown precursors. In some aspects, visualization of the target tissue is performed using one or more known methods, including those previously described herein. The visualization step assists with identifying the location of the target tissue to which the cold solution will be administered, determining whether the target area should be adjusted, and/or monitors the efficacy of the treatment.

A pre-treatment and/or post-treatment step may include treatment utilizing a topically applied cold solution. A pre-treatment and/or post-treatment step may include implanting a balloon or a custom made 3-D printed hollow implant comprising a cold solution, wherein the balloon or implant may provide a continuous or variable administration of the cold solution. A pre-treatment and/or post-treatment step may include implanting a pharmacological device, which delivers a suitable pharmacological agent continuously, or at pre-determined intervals, including those previously described herein.

A pre-treatment step may include pre-activation of adipose tissue by activating local sympathetic input to fat using an energy based device, including but not limited to a transcutaneous electrical nerve stimulation (TENS) device. A pre-treatment step may include pre-activation of adipose tissue through administration of a contrast agent and/or a pharmacological agent as disclosed previously herein. A pre-treatment step may include pre-activation through topical cooling, e.g., topical administration of the cold solution. A pre-treatment step may include pre-activation through topical administration of the cold solution, following by imaging through any known imaging method. The imaging method may be selected from the group of thermal imaging, CT, PET and MRI, which focus on activity such as rate of blood flow or metabolism, and accordingly are particularly applicable to analyzing the activation and proliferation of brown fat cells.

In some aspects, an incision is made to provide access to the target area, followed by administration of the cold solution, e.g., via injection, to the target area. The incision may also be used for imaging, in order to study and select the target area, and/or to monitor and evaluate the treatment efficacy. Such a treatment is helpful for difficult-to-reach adipose tissue and/or visceral fat.

In some aspects, the cold solution is administered to the subject via a port, which is implanted at or near the treatment site of the subject. The port may be utilized to administer the cold solution continuously, or at pre-determined intervals.

An implantable thermoelectric cooler may be implanted in a pre-treatment, treatment, and/or post-treatment step. A long lasting drug eluting implant may be implanted, wherein the implant comprises a pharmacological agent selected from those previously described herein, in a pre-treatment, treatment and/or post-treatment step.

In one aspect, a pre-treatment step includes activating brown adipocytes by topical cooling, such as with a 1 hour cooling vest, or topical administration of the cold solution, followed by administration of a 133 receptor agonist, e.g., 200 mg mirabegron. After the pre-treatment step, visualization is performed through imaging using 18F-FDG-PET/CT, thermal imaging, magnetic resonance imaging, ultrasound imaging, or the like. After visualization/imaging, the cold solution is injected into brown adipose tissue in which brown adipocytes have been pre-activated.

In one aspect, a pre-treatment step includes visualizing brown adipocytes through imaging using any known method in the art, including ¹⁸F- or ¹⁴C-fluorobenzyltriphenyl phosphonium, (S,S)—O-[¹¹C]methylreboxetine, ¹¹C-methylreboxetine CT/PET, ¹⁸F-FDG PET, ¹⁸F-FDG PET/CT, thermal imaging, MM, and ultrasound. Following the visualization/imaging, the cold solution is injected into brown adipose tissue in which the brown adipocytes are unactivated, in order to activate the brown adipocytes. The cold solution may also comprise one or more pharmacological agents selected from the group previously set forth herein, such as a β-receptor agonist.

In one aspect, a pre-treatment step includes visualization of sympathetic innervation of adipose tissue through imaging using any known method in the art, including (S,S)—O-[¹¹C]methylreboxetine PET, which binds to norepinephrine (NE) receptor, ¹¹C-meta-hydroxyephedrine. After visualization/imaging, the cold solution is injected into area(s) with sympathetic innervation, which is an indicator of the presence of beige and/or brown adipocytes. The cold solution may also comprise one or more pharmacological agents selected from the group previously set forth herein, such as a β-receptor agonist, or a PPARγ agonist such as rosiglitazone.

In one aspect, a pre-treatment step includes identification of brown and/or beige adipocytes using thermal imaging or based upon anatomic location. Following the pre-treatment imaging, the cold solution is injected into the brown and/or beige adipocytes.

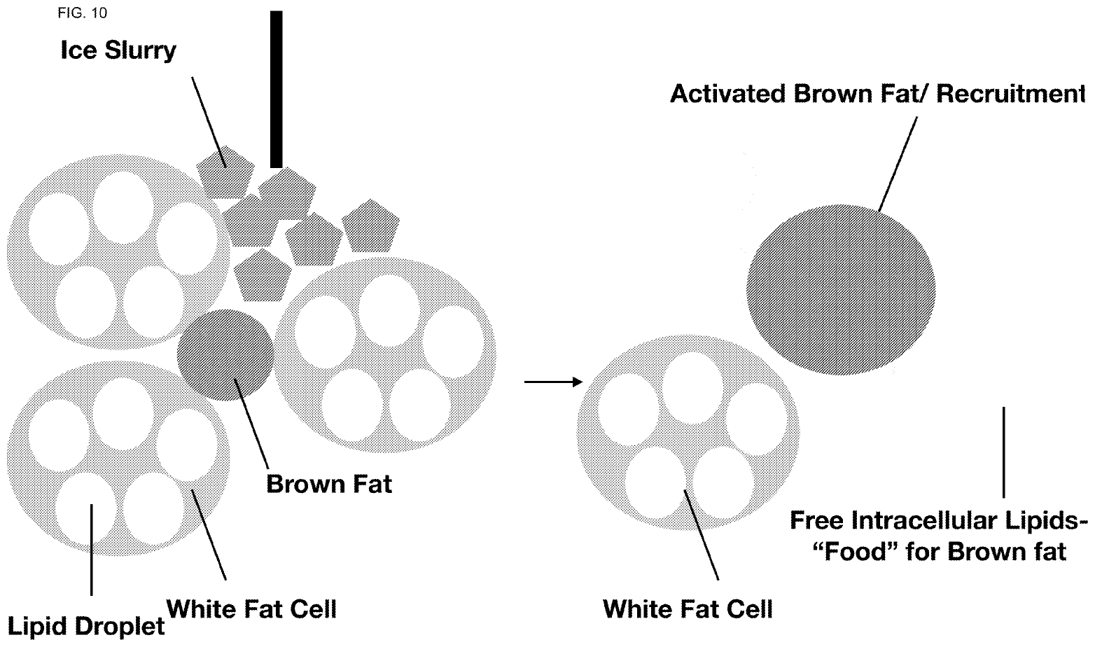

In one aspect, the desired location of white precursors, i.e., the target area, is determined prior to treatment with the cold solution based upon known anatomical locations, and/or through imaging using a known imaging method. After identification of the desired target area comprising white precursors, the cold solution is administered to target area, and optionally the surrounding area. The administration of the cold solution to the target area stimulates white precursors present therein and directs differentiation from the white precursors into the more metabolically beneficial beige fat. Additionally, administration of the cold solution to the location of the white precursors and optionally the surrounding area has the additional benefit of initiating cryolipolysis of white fat cells in the treatment site and surrounding area. In addition, as shown in FIG. 10, when cryolipolysis is initiated, fatty acid/lipid droplets are released from the white fat, where the droplets act to activate brown or beige fat in proximity to the white fat by stimulating expression of UCP1.

In one aspect, the desired location of beige and/or brown precursors, i.e., the target area, is determined prior to treatment with the cold solution based upon known anatomical locations and/or through imaging using a known imaging method. After identification of the desired target area comprising beige and/or brown precursors, the cold solution is administered to the target area, and optionally the surrounding area. The administration of the cold solution to the target area comprising beige and/or brown precursors stimulates adipogenesis from the beige and/or brown precursors to beige and/or brown fat cells, respectively. Additionally, administration of the cold solution to the location of the beige and/or brown precursors and optionally the surrounding area has the additional benefit of initiating cryolipolysis of white fat cells in the and around the treatment site.

Beige fat cells are surrounded by white fat cells. Accordingly, in one aspect, the desired location of beige fat cells is determined prior to treatment with the cold solution based upon known anatomical locations and/or through imaging using a known imaging method. After identification of the desired location comprising beige fat cells, the cold solution is administered to the target area, which is the white fat cells which surround the beige fat, in order to initiate cryolipolysis of white fat cells, and to effect transdifferentiation from other white fat cells to the more metabolically beneficial beige fat cells. In some aspects, a first cold solution is administered to initiate cryolipolysis of the white fat cells surrounding the beige fat cells, and a second cold solution is administered to effect transdifferentiation of the white fat cells remaining around the beige fat cells into additional beige fat cells. The first and second cold solution may be identical, or may have different compositions and properties.

In some aspects, the administration of the cold slurry to a treatment site and surrounding area of a subject has additional benefits. For example, exposure to cold through administration of the cold solution alone, or in combination with other known methods, such as topical cooling of the skin, increases the metabolism of the subject, improves blood sugar of the subject and may result in weight loss.

A system for the methods described above may comprise an imaging device, a delivery device for delivering a cold solution comprising liquid water and/or solid particles to a treatment site of a subject, and a cold solution supply source configured to supply the cold solution. The cold solution supply source is any suitable supply source, including but not limited to a pump system that generates flow of the cold solution through the delivery device, for example, via a syringe. The delivery device is any suitable device for administering the cold solution to a treatment site of a subject, including but not limited to a cannula such as a needle, a needle array, e.g., an expandable needle array, a fenestrated needle, an implantable balloon or an 3D printed custom implant. The imaging device may be any suitable imaging device or procedure, including but not limited to an MM, CT, ultrasound, PET, visual assessment, thermal imaging device, 3D imaging device, or any other device related to a known imaging procedure such as those described herein.

A system for the methods described above may comprise an imaging device, a first delivery device for delivering a first cold solution comprising liquid water and/or solid particles to a treatment site of a subject, and optionally a second cold solution comprising liquid water and/or particles to a treatment site of a subject, optionally a second delivery device for delivering the second cold solution comprising liquid water and/or solid particles to a second treatment site of the subject, a first cold solution supply source configured to supply the first cold solution and optionally the second cold solution, and optionally a second cold solution supply source configured to supply the second cold solution. Each cold solution supply source is any suitable supply source, including but not limited to a pump system that generates flow of the cold solution through the delivery device, for example, via a syringe. Each delivery device is any suitable device for administering the cold solution to a treatment site of a subject, including but not limited to a cannula such as a needle, a needle array, e.g., an expandable needle array, a fenestrated needle, an implantable balloon or an 3D printed custom implant. The imaging device may be any suitable imaging device or procedure, including but not limited to an Mill, CT, ultrasound, PET, visual assessment, thermal imaging device, 3D imaging device, or any other device related to a known imaging procedure such as those described herein.

In some aspects, a method of treatment comprises treatment of one or more layers of superficial adipose tissue, including superficial (sSAT) and deep (dSAT) layers of adipose tissue. The methods of treatment described herein may be used independently, in combination, or together with a treatment of superficial and/or deep adipose tissue, such as the methods described in International Application No. PCT/US2018/054834, filed Oct. 4, 2019, U.S. Provisional Application No. 62/953,272, filed Dec. 24, 2019, and International Application No. PCT/US2019/055605, filed Oct. 10, 2019, the contents of which are incorporated herein in their entirety.

The cold solution may be utilized to effect tissue engineering in vivo or ex vivo, such that improved fat cells may be transferred into the subject. The improved fat cells may be implanted in the location from where the harvested cells were obtained, or may be implanted in a different location of the subject

For in vivo applications, the cold solution may be administered to a treatment site containing beige fat cells, in order to effect transdifferentiation of the beige fat cells into brown fat cells, thus improving the metabolic properties of the cells. Prior to the administration of the cold solution, the location of the beige fat cells is visualized and identified using a known visualization or imaging method.

For ex vivo applications, tissue comprising white fat cells, white precursors, beige fat cells and/or beige precursors, may be harvested from the subject by any method known in the art, including but not limited to liposuction. A scaffold may be used during the tissue harvesting, in order to improve the viability of the fat cells to be transferred. The scaffold may comprise an agent to promote angiogenesis and/or comprise pores. In some aspects, the scaffold may be biodegradable, to allow for implantation into the subject in combination with the improved fat cells through integrated fat transfer. Prior to the harvesting, the location of the desired fat cells is visualized and identified using a known visualization or imaging method. The harvested fat cells are treated with the cold solution, in order to improve the metabolic properties thereof. The treated, harvested fat cells are then implanted into the subject in the location where the cells were removed, or in another location of the subject, such as any location in the subcutaneous fat layer.

In one aspect, a method may include a pre-treatment comprising selecting a target area comprising brown fat based upon known anatomical locations and/or through imaging, followed by activation of the brown fat in the target area by administration of the cold solution through the methods previously described herein. Following the pre-treatment, a sample of the brown fat in the target area is removed through any method known in the art. The brown fat cells are separated from other extracted cells via any method known in the art. A cold growth culture media is prepared by combining the cold solution with a known cell growth media to create a cold growth culture media for use in the culture of the extracted cells. The extracted brown cells are expanded ex vivo using the cold growth culture media in any cell growth method which is appropriate for adipocytes, to create newly grown cells. The extracted brown fat cells and newly grown cells are then injected into the target areas, thus immediately increasing the volume of brown adipocytes, and improving metabolism.

In another aspect, after identifying the treatment site through any known visualization method, the cold solution is administered to the treatment site to induce cryolipolysis, or to improve the metabolic nature of the cells through transdifferentiation into a different type of fat cell, and/or to effect change from a fat cell precursor into a white fat cell, a beige fat cell or a brown fat cell. After treatment with the cold solution, the desired fat cells are harvested from the subject by any method known in the art, and are transferred to another location of the subject.

Human infants possess brown fat cells, which assist with the important function of maintaining body temperature after birth. However, an infant born prematurely may not have sufficiently developed the requisite amount of brown fat cells, and thus may struggle with temperature regulation. Accordingly, there is a need to increase the amount of brown fat cells in prematurely born infants, such as by transplantation into the infant. The brown fat cells may be harvested from a donor, or may be developed from tissue from the infant, in any method described above. In another aspect, the cold solution may be administered to the infant, in order to activate existing brown fat cells, or to effect change from existing white precursors, beige precursors, white fat cells and/brown precursors to more metabolically active beige or brown fat cells. Prior to administration of the cold solution, visualization by imaging may be performed to determine the optimal location(s) for administration of the cold solution. In another aspect, the location of the administration of the cold solution may be selected based upon general knowledge regarding location of the desired tissue and/or cells.

In some aspects, prior to, during, or after a method of treatment according to the invention, the cold solution is administered to the subject in order to reduce inflammation, topically or via injection.

In order to demonstrate the effects of the compositions and methods of the present invention, the following experiments are performed.

A patient group of 10 to 20 subjects is provided. In a first experiment, the subjects of the patient group are treated by image guided subcutaneous injection of a cold solution into one or more of supraclavicular brown fat, axillary brown fat and neck brown fat. In a second experiment, the subjects of the patient group are treated with multiple treatment cycles, i.e., multiple injections of a cold solution into one or more of supraclavicular brown fat, axillary brown fat and next brown fat, wherein the timing between the injections may vary according to experimental protocol. Each of the subjects is subjected to imaging pre-treatment and post-treatment, for each treatment, to determine efficacy of the treatment. Measurements of each subject are obtained, including but not limited to brown fact activation/volume, weight loss, basal metabolic rate (BMR), glucose tolerance test, and inflammatory/metabolic biomarkers. The amount of cold solution to be administered depends on many factors, including but not limited to the amount of fat cells to be treated, the specific target tissue, the goal of the treatment, and the number of treatments to be administered.

A study is performed to assess activation and expansion of brown and beige adipocytes, and induction of cryolipolysis of white adipocytes after administration of a cold solution. The study utilizes 24 wild type, 12 week male C57BL/6J mice. The mice are separated into the following test groups, wherein each test group includes 3 mice: Day 1 (control), Day 1 (ice slurry), Day 3 (control), Day 3 (ice slurry), Day 7 (control), Day 7 (ice slurry), Week 8 (control) and Week 8 (ice slurry). The control is a cold solution comprising no solid ice particles. The ice slurry is a cold solution comprising about 2% to about 70% solid ice particles. Each mouse is treated with 10 mL of the control or the ice slurry. The treatment comprises injection of a part or all of the control or the ice slurry into an inguinal fat pad of the mouse, with any remainder (between about 9 mL and about 1 mL) applied topically to and around the injection site. Additional studies are conducted using varied amounts of the control and the ice slurry, in line with the ranges disclosed herein. Thermal images are obtained of each injection site and its surrounding area immediately after injection. The mice are sacrificed at Day 1, 3, 7 or Week 8, depending upon the test group. After sacrifice, photographs are obtained of the skin, as well as exposed fat pads of the mice. Tissue histology of the skin, inguinal fat pads, intrascapular fat pads and underlying muscle of the sacrificed mice is obtained. Particularly, the following histology is obtained for each sacrificed mouse: biopsy of skin anterior to the injected inguinal fat pad using hematoxylin and eosin stain (H+E) and an unstained biopsy of skin anterior to the injected inguinal fat pad; unstained biopsies of each side of the injected inguinal fat pad; an unstained biopsy of the intrascapular fat pad (removed, uncooled fat); and an unstained biopsy of muscle underlying the inguinal fat pad. The biopsied sample(s) are further studied for the presence of one or more of UCP1, CD31, VEGF, markers of cryolipolysis, markers of apoptosis, immune infiltrates, and collagen. The fat pad is then excised and prepared for rtPCR (reverse transcription polymerase chain reaction) for UCP1, Pgc1a, Ppara, and Cidea, to determine if local markers of brown fat regulation are unregulated, and to determine if there is an effect on remote fat.

Information regarding blood flow in and around the injection site may be obtained using an ultrasound sensor comprising ultrasound transducers which is applied to the skin, and measures blood flow within the body. An exemplary ultrasound sensor is described at https:www.medgadget.com/2021/07/ultrasound-patch-monitors/blood-flow.html. Evidence of an increase in blood flow demonstrates thermogenic tissue activation.

A single treatment of the ice slurry injection is sufficient to induce cryolipolysis of white adipocytes while simultaneously activating and triggering expansion of thermogenic fat (brown/beige fat). Evidence of this effect will be seen on histology with increased presence of brown/beige fat cells, as well as the characteristic histologic response of cryolipolysis, including cold-induced panniculitis in the fat tissue, a reduction in the number and size of white adipocytes, and increased collagen. Further, activation and expansion of brown fat will be assessed through rtPCR of genes specific to thermogenic fat, such as UCP1. The cold temperatures to activate brown fat have previously been substantially warmer than those required to induce cryolipolysis. The injection of the ice slurry with dramatic cooling capacity, rapid rate of cooling, and temperatures in the −10 to 4° C. range can simultaneously remove metabolically unfavorable white adipose tissue via cryolipolysis, while stimulating metabolically favorable thermogenic adipose tissue, which will contribute to an overall improvement in the metabolic profile of subcutaneous adipose tissue.

The methods according to the invention may be useful in many scenarios. In one aspect, a method may be used to increase the amount of brown fat cells in an infant born prematurely. In another aspect, a method may be used to increase the metabolism of a subject, by increasing the number of metabolically active fat cells, e.g., brown fat cells and beige fat cells. In another aspect, a method may be used to improve the health of a subject, by initiating cryolipolysis of lipid rich white fat cells, initiating change from a fat cell precursor to a metabolically active fat cell, and/or inducing transdifferentiation from a white or beige fat cell into a more metabolically active fat cell. In another aspect, a person in the military may benefit from methods according to the invention, by jump-starting a health regimen aimed at fat reduction, improved metabolic performance, and increased muscle mass.

The methods of the invention may be utilized in combination with one or more additional modalities, as described in U.S. Provisional Application No. 63/035,139, filed Jun. 5, 2020, the contents of which are incorporated herein in their entirety. The one or more additional modalities may be administered in one or more of the pre-treatment, treatment or post-treatment sessions, or may occur before, between, or after one or more of the sessions.

The one or more additional modalities include, but are not limited to, energy, surgery, nutrition and/or wellness, exercise, and aesthetic, chemical and/or biological treatments. A modality which utilizes energy may include thermal energy, radiant energy, chemical energy, electrical energy and/or mechanical energy. In some aspects, the additional modality can be used to augment or supplement the treatment with cold solution.

In some aspects, thermal energy may be utilized to increase the temperature at or near the injection site and/or the treatment site. Thermal energy may be administered by any method known in the art, including but not limited to, hot or warm cloth, hot or warm water bottle, hot or warm bath, ultrasound, heating pad, heat therapy wraps, hydrocollator heat packs, and injection of a warm solution. The increase in temperature at or near the injection and/or treatment site may alter the physical characteristics of the tissue in and around the site, thus increasing fat reduction and improvement of the appearance of the skin. In some aspects, the tissue may be thinned and/or have increased flexibility. In some aspects, the blood flow at or near the site may increase, thus improving oxygenation and wound healing. In some aspects, the administration of thermal energy may reduce pain and/or inflammation at or near the site. In some aspects, the administration of thermal energy may activate a pharmacological agent which may be administered before, during, or after the administration of the cold solution.

In some aspects of the invention, radiant energy may be utilized through any device such as a laser and/or method known in the art, including but not limited to utilization of solar energy, visible light, infrared waves, radio waves such as radiofrequency, ultraviolet waves, X-rays, microwaves, and/or radium, in the treatment of the subject. In some aspects, a photosensitizer and light source can be administered to the treatment area, for example to improve the appearance of skin such as to reduce stretch marks. Examples of photosensitizers include but are not limited to 5-aminolevulinate, porphyrins, chlorins, bacteriochlorins, phthalocyanines, phenothiazinium salts, rose Bengal, squarines, BODIPY dyes, phenalenones, ruthenium compounds, rhodium compounds, hypericin, hypocrellin, riboflavin, and cur cumin. Examples of light sources include but are not limited to light-emitting diodes, lasers and intense pulsed light. In some instances, the radiant energy may be utilized to treat an area of the subject, or to diagnose and study an area of the subject.

In some aspects, chemical energy may be utilized through any device and/or method known in the art, in the treatment of the subject. In some aspects, chemical energy is administered by administering one or more substances to the treatment site, wherein the one or more substances cause an exothermic reaction thereby heating the treatment site. In some aspects, one or more substances can be administered to the treatment site where the one or more substances cause an exothermic reaction thereby heating the treatment site. In some aspects, when fat cells are subjected to cold, such as through the administration of a cold solution, the fat cells release energy as heat, thus further aiding in the reduction of fat.

In some aspects, electrical energy may be utilized through any device and/or method known in the art, including but not limited to electrical stimulation devices and electromagnetic devices, such as an electronic muscle stimulator or a transcutaneous electrical nerve stimulator (TENS), in the treatment of the subject. In some aspects, electrical energy may be administered in order to relax and/or tone muscles, increase blood circulation, manage pain, improve wound healing, and/or assist with drug delivery, for example, in the process of iontophoresis.

In some aspects of the invention, mechanical energy may be utilized through any device and/or method known in the art, in the treatment of the subject. In some aspects, mechanical energy may be administered through ultrasound, massage, vibration, pulsation and/or compression.

In some aspects of the invention, mechanical energy may be utilized through fractional wounding, wherein micro-holes are drilled in or around the treatment site, in order to stimulate collagen production and/or deliver the cold solution to the subject. A device containing a pre-fabricated array, for example, an array of needles or cannulas, may be utilized to drill the micro-holes in an appropriate pattern, wherein the pattern is in an appropriate shape for the size and shape of the administration site, and may include a grid, wherein the grid is a square-shape, a rectangular-shape, a circular-shape, or a triangular shape, a plow-shape, a fan-shape, a combination thereof, or a modification thereof.

In some aspects of the invention, mechanical energy may be utilized by administering filaments, wherein the filaments may be administered via a staple gun. In some aspects of the invention, the filaments are biodegradable, and may optionally be loaded with a pharmacological agent.

In some aspects of the invention, mechanical energy may be utilized by administering resorbable sutures in combination with the cold solution to the subject. In particular, a device is generated by placing one or more resorbable sutures in a mold, for example, a cannula, and placing a solution comprising water and optionally one or more additives in the mold, prior to cooling the mold and components therein. After achieving the desired ice coefficient through cooling, the cold solution further comprising one or more resorbable sutures is removed from the mold in an appropriate manner, and administered to the desired treatment site through an appropriate method, for example, via injection. After administration, the cold solution melts, through any suitable active or passive measure. The one or more resorbable sutures remain at or near the treatment site until resorption acting as an irritant to the tissue surrounding the treatment site. A cosmetic benefit occurs due to the increased collagen produced around the treatment site due to the presence of an unknown irritant, specifically the one or more resorbable sutures. The increased collagen promotes cell renewal, which provides cosmetically appealing results. In some aspects, the sutures may be provided in a pattern suitable to the treatment site. For example, when treating the chin and neck area, the sutures may be administered in an arc pattern just below the jawline, thus providing minimally visible suture sites.

In some aspects, mechanical energy may be utilized by administering ultrasound, for example via delivery device comprising a transducer. In some aspects, an ultrasound needle or catheter can be used to deliver energy, for example, the ultrasound catheter disclosed in Burdette, Everette et al., (2010), The ACUSITT Ultrasonic Ablator: The First Steerable Needle with an Integrated Interventional Tool, Proceedings of SPIE—The International Society for Optical Engineering, 7629. 10.1117/12.845972, the contents of which are incorporated by reference in its entirety.

A modality which utilizes surgery may include any appropriate surgical procedure known to those skilled in the art, including but not limited to filler injection (including but not limited to fat, collagen, hyaluronic acid, and any compositions disclosed in U.S. Provisional patent application Ser. No. 63/001,889 filed on Mar. 30, 2020 which is incorporated herein by reference in its entirety), liposuction, abdominoplasty, gluteoplasty, brachioplasty, thighplasty, lower rhytidectomy, mentoplasty and bariatric surgery.

A modality which utilizes nutrition and/or wellness may include monitoring and/or adjusting daily food intake, type of food and/or supplements, and/or calorie consumption of the subject to reduce fat. A nutrition and/or wellness modality may also include one or more of nutritional analysis, nutrition coaching, lifestyle coaching, weight loss, a personalized food plan/guide, personalized recipes, a personalized nutrition plan/guide, meal box service, grocery delivery, a farm/meat share subscription, regenerative medicine, traditional aesthetic medicine, mindfulness, sleep tracking, e.g., sleep cycle, assistance of a sleep coach, light-based smart-home technologies, e.g., hue-adjusted smart lights, smart-shades, integration with sleep data to optimize wake up and go to sleep timeframes, blue light management, obstructive sleep apnea therapy, indirect data metrics, including but not limited to meal purchase, use of refrigerator artificial intelligence, career planning and/or coaching, acupuncture, energy-based therapies, reiki, use of informational websites, use of smart technologies, e.g., Apple Health app, Apple Activity app, Apple fitness tracking, Calm app, Map My Walk app, Headspace app, MyFitnessPal, and Google Fit, use of a health cloud, e.g., Sales Force 360 CRM, spiritual guidance, participation in faith based/spiritual communities, participation in faith based/spiritual experiences, financial planning, retirement planning, finance tracking, e.g., earnings and spending, and travel planning.

A modality which utilizes exercise may include implementing, or adding to, an exercise regimen to reduce fat, increase muscle and/or maintain weight and muscle content. An exercise modality may also include one or more of personal training or coaching, physical therapy, at home workout, muscle stimulation, supplements, e.g., personalized supplements, meditation, yoga and tracking performance metrics.

A modality which utilizes self-optimization may include one or more of cosmetics including but not limited to hair products, skin products and nail products; aesthetic medicine approaches; a skin care regimen or treatment including but not limited to skin typing, personalized skin care regimen, topicals, skin tightening, cryangiogenesis, wrinkle management, treatment of dark spots, treatment of hyperpigmentation, texture, hydration, environmental stressor protection, e.g., sun care, pollution prevention, treatment of non-facial skin including but not limited to scars, striae, cellulite, skin laxity, keratosis pilaris, hyperhidrosis, folliculitis, and intertrigo; and a cosmetic procedure such as a non-invasive, minimally invasive or invasive procedure.

A chemical and/or biological modality may include treatment of the subject using small molecules, large molecules, mid-size molecules, protein degraders, antibody drug conjugates, gene therapy and/or molecular probes. Such a treatment may include the administration of one or more a pharmacological agent which may augment the treatment of brown fat. The administration of the pharmacological agent may be in any suitable method. In some aspects of the invention, the pharmacological agent includes, but is not limited to the pharmacological agents previously disclosed herein. In some aspects of the invention, the one or more pharmacological agents may be administered prior to, concurrently with, or subsequent to administration of the cold solution. When the one or more pharmacological agents are administered concurrently with the administration of the cold solution, the one or more pharmacological agents may be administered independently from the cold solution, or may be present in the cold solution.

A modality that augments or supplements the treatment using a cold solution, e.g., one or more injections of cold solution, can enhance the cold solution treatment, reduce side effects of the cold solution treatment and/or improve the outcome of the cold solution treatment. Modalities to enhance the cold solution treatment include but are not limited to administering an anesthetic prior to the cold slurry treatment; a modality to prevent leakage of the melted slurry, e.g., a dressing such as a superabsorbent polymer/water-absorbing polymer dressing such as a hydrogel dressing, gauze dressing, alginate dressing, hydrofiber dressing, foam dressing, medical bandage or adhesive; a modality to keep the subject warm before, during or after the treatment, e.g. a warming blanket, a non-contact thermal light source such as an infrared mask, hot water bottle, heating pad, heated treatment table, and a mild capsaicin cream (to increase blood flow and thus the perception of warmth); and a modality to minimize any noise created by the device during treatment, e.g., ear plugs, noise cancelling headphones or a wearable such as a foam padded hat. Modalities to reduce side effects include but are not limited to modalities to decrease bruising and/or inflammation caused by an injection, e.g., a topical comprising arnica and/or menthol; and a modality to improve healing and/or reduce scarring at an injection site, e.g., a topical such as a retinoid, corticosteroid cream, onion extract cream, petrolatum ointment, a dressing such as a silicone dressing, and a mechanical modality such as a massage or vibration device to relieve tension at the injection site. Modalities to improve outcomes include but are not limited to a modality to break up the fibrous septae in advance of a treatment, e.g., a mechanical or thermal device as described herein; a modality to heating the treatment site before the treatment (either at home or in the treatment room), e.g., a warming blanket or thermal energy described herein; a modality to enable visualization during the procedure as described herein, modality to further break up fibrous septae and/or ice crystals post-treatment as described herein; and a modality to ensure symmetry post treatment, e.g., a compression band.

An exemplary method of treatment according to the invention includes creating a treatment plan, followed by pre-treatment, treatment and post-treatment.

In an aspect of the invention, a pre-treatment comprises heating, disrupting and/or preparing the treatment site immediately prior to administration of the cold solution. In some aspects, a sheath, e.g., a cannula or needle, comprising a lumen is inserted into the treatment site, and a device, e.g., an energy device, is inserted through the sheath. The energy device can be any device described herein. In some aspects the energy device comprises one or more of an ultrasonic cutting tip, a resistive heater or a light source.

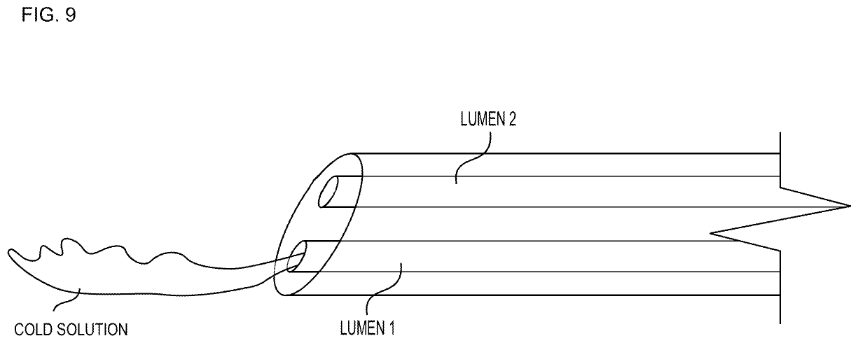

In some aspects, a sheath comprising more than one lumen (a multi-lumen sheath) is inserted into the treatment site, wherein a device, e.g., an energy device and the cold solution are administered via the more than one lumen. FIG. 9 demonstrates a sheath comprising a first lumen and a second lumen, wherein a cold solution may be administered to the treatment site via the first lumen, and an energy device can be inserted into the second lumen for administration to the treatment site. Any number of working channels (or lumens) can be included, for example, two, three, four, five or six lumens, each configured to receive a device. In some aspects, a visualization device can be inserted in a lumen.

A system for the method described above comprises a sheath comprising a lumen, an energy device configured to supply energy to a subject such as a TENS device, and a cold solution supply source configured to supply a cold solution comprising liquid water and/or solid ice particles, the cold solution to be administered to a treatment site of the subject via the lumen. In another aspect, the sheath comprises a first lumen and a second lumen, wherein the energy device is configured to administer energy to the subject via the first lumen, and the cold solution is configured to be administered to the subject via the second lumen. The cold solution supply source is any suitable supply source, including but not limited to a pump system that generates flow of the cold solution through the sheath, for example, via a syringe.

In some aspects, an energy device is inserted into the treatment site through the lumen in the sheath, and directed toward the treatment site, in order to locally heat the treatment site, disrupt fascia between septae, cut tissue, provide illumination, provide imaging, collect data, and/or maximize the temperature difference experienced by the treatment site (contrast cryolipolysis) before and after administration of the cold solution. In some aspects, an energy device is administered directly into the treatment site. The energy device can comprise any of the energy devices described herein. In some aspects, the energy device is an ultrasonic device, a resistive heater, an ultrasonically actuated cutting tip, a light guide or an optical fiber.

When using a single lumen sheath, immediately prior to administration of the cold solution, the energy device may be removed from the sheath or cannula, followed by administration of the cold solution through the open lumen. In some aspects, the cold solution delivery device described above includes an ultrasound transducer which may generate heat and mechanical energy. In some aspects, generation of heat and mechanical energy will be located in a separate part of the delivery device, so as to avoid undesired heating of the cold solution. When utilizing a multi-lumen sheath, the energy device may remain in place in the first lumen, and the cold solution may be administered through the second lumen.

In some aspects, radiant energy may be administered to the treatment site through the single lumen sheath or the multi-lumen sheath, or directly, in order to preheat the treatment site prior to administering the cold solution. For example, radiant energy may be administered to the treatment site prior to administering a cold solution, e.g., a slurry or a substantially solid solution, thus maximizing the temperature difference experienced by the tissue (contrast cryolipolysis), and augmenting the effects of the cold solution.

In some aspects, a cold solution is administered to the treatment site to provide cooling in order to increase the tolerability, and to limit thermal diffusion of radiant energy sources for skin treatment. For example, the cold solution may be administered to the treatment site via injection, followed by treatment of the skin at the administration site with a heat-based modality, in order to treat brown fat. A cold solution delivery device may be coupled to the heating device. Such a method may enable treatment with higher levels of energy, because a cooling source is placed distal to the treatment area. A substantially solid solution may be applied on the skin to actively cool the administration site and surrounding area, followed by administration of light. The administration of the cold solution on the skin serves as a cooling source, e.g., numbing agent, to increase the tolerability of further administrations to the treatment site, e.g., laser treatment, and potentially increases the energy of traditional thermal methods of skin tightening and remodeling, including but not limited to laser, radio frequency, ultrasound skin tightening.

In an aspect of the invention, the cold solution and a secondary solution comprising a second agent, e.g., target molecules, are simultaneously delivered to the treatment site during administration, and the second agent is activated secondarily, for example, after the original administration and/or in a home environment. FIG. 9 demonstrates an example of a multi-lumen sheath, wherein the cold solution is administered via a first lumen, and the secondary solution is administered via a second lumen. A multi-lumen sheath is inserted into the treatment site, the cold solution is delivered through a first lumen, and a second agent, such as gold particles (e.g., gold microparticles or nanoparticles) in suspension, is delivered through a second lumen. At some period of time after the administration of the cold solution and the second agent, for example, after the cold solution melts, the second agent remains distributed over the area treated by the cold solution and may be externally activated, e.g., topically, to product combinatory effect. For example, light may be used to target heating of gold nanoparticles which were administered as the second agent. In some aspects, the second agent can comprise silver particles (e.g., silver microparticles or nanoparticles) or microbubble enclosed particles. In some aspects, the particles enclosed in microbubbles can be activated via an ultrasound device.

A system for the method described above comprises a sheath comprising a first lumen and a second lumen, a cold solution supply source configured to supply a cold solution comprising liquid water and/or solid ice particles, the cold solution to be administered to a treatment site of a subject via the first lumen, and a secondary solution supply source configured to supply a secondary solution comprising a second agent, the secondary solution to be administered to a treatment site of a subject via the second lumen. The second solution supply source is any suitable supply source, including but not limited to a pump system that generates flow of the second solution through the sheath, for example via a syringe.

In an aspect of the invention, a cold solution is administered to a treatment site, followed by use of an additional modality, e.g., a laser, to topically heat the treatment site. The additional modality may be selectively applied, such that the cold solution is melted in selected areas. In some aspects, the melted areas may result in decreased therapeutic effect. In some aspects, the use of selective melting may provide a desired contouring effect in or around the treatment site.

In an aspect of the invention, a pre-treatment comprises heating, disrupting and/or preparing the treatment site immediately prior to administration of the cold solution, wherein the pre-treatment utilizes a fenestrated needle. In one aspect, the fenestrated needle is inserted into the treatment site, followed by injection of a target molecule solution, such as gold particles, silver particles, microbubble enclosed particles, or another chemical component, into the treatment site. The target molecule solution is supplied by a target molecule solution supply source, which is any suitable supply source, including but not limited to a pump system that generates flow of the target molecule solution through the fenestrated needle. After injection of the target molecule solution, the fenestrated needle is removed. In another aspect, after injection of the treatment site, energy is administered through the lumen of the fenestrated needle. The energy source may be an ultrasonic device, a resistive heater, an ultrasonically actuated cutting tip, a light guide or an optical fiber, which may locally heat the treatment site, disrupt fascia between septae, cut tissue, provide illumination, provide imaging, collect data, and/or maximize the temperature difference experienced by the treatment site (contrast cryolipolysis). For example, see FIG. 40B, which demonstrates a target molecule solution being light activated through the fenestrations of the fenestrated needle. Prior to administration of the cold solution, the energy device is removed. In one aspect, the cold solution is administered via a delivery device attached to the fenestrated needle. In another aspect, the fenestrated needle is withdrawn, and the cold solution is delivered via a delivery device.

In some aspects, pre-treatment includes obtaining measurements of the treatment site and/or the surrounding area through imaging, including but not limited to Magnetic Resonance Imaging (MRI), Computed Topography (CT), ultrasound, Positron Emission Tomography (PET), 3D imaging, and combinations thereof. Imaging may be used in the absence of, or together with, an incision near or at the treatment site. In another aspect, measurements of the treatment site and/or the surrounding area may be obtained through a computer or artificial intelligence system, which contains data obtained from the subject to be treated and/or data obtained from multiple subjects.

In some aspects, pre-treatment also include a heat application at or near the injection site, which may improve cryolipolysis due to the difference in temperature between the warmed injection site and the cold solution to be administered, i.e., contrast cryolipolysis.

In some aspects, pre-treatment also includes selective disruption of fibrous tissue at the treatment and/or injection site and the surrounding area, thus enabling a smoothing effect in addition to the fat reduction. The selective disruption of fibrous tissue may occur through any suitable method, including but not limited to, mechanical vibration, application of heat, and/or topical or subcutaneous administration of energy activated nanoparticles, e.g., gold or silver. The selective disruption of fibrous tissue may also occur by administration of a cold solution, which may be utilized to mechanically disrupt fibrous tissue to break up compartments found within the subcutaneous fat, allowing the subcutaneous fat to spread and create a visually smoother appearance, for example in the treatment of cellulite, as described in U.S. Provisional Application No. 62/953,272, previously incorporated by reference, and in International Patent Application Ser. No. PCT/US2017/059947 filed on Nov. 13, 2017, the entirety of which is incorporated herein by reference.

In some aspects, pre-treatment also includes fractional wounding by drilling holes at or near the treatment site, in order to disrupt fibrous tissue and/or stimulate collagen production.

In some aspects, treatment includes injection of a cold solution to the subject, in any suitable amount, in the methods described above, optionally together with one or more pharmacological agents, as described above, or energy activated nanoparticles, such as gold or silver. The cold solution, one or more pharmacological agents, and/or energy activated nanoparticles may be administered simultaneously and/or separately. If the cold solution, one or more pharmacological agents and/or energy activated nanoparticles are administered separately, the cold solution may be administered before, after, or before and after the administration of the one or more pharmacological agents and/or energy activated nanoparticles.

In some aspects, treatment includes heat application in order to improve cryolipolysis, application of mechanical energy, such as vibration, massage, pulsation, and/or compression, to assist with cell death after cold solution is injected, fractional wounding to deliver cold solution, and/or administration of resorbable sutures.

In some aspects, post-treatment includes one or more of magnetic muscle stimulation (MMS) in order to develop/improve muscle tone, compression, thermal compression, cool/cold compression, activation of previously deposited energy activated nanoparticles, nutrition planning and monitoring, exercise, topical application of a microneedle patch to allow transdermal delivery of one or more pharmacological agents, as described above, and/or the collection of samples, including but not limited to blood and interstitial fluid, which may utilized for data collection and analysis, collection of data using a computer program or application, and/or fractional wounding in order to stimulate collagen production.

All documents, books, manuals, patents, published patent applications, and other reference materials cited herein are incorporated by reference in their entirety.

While the invention has been described with reference to certain particular aspects thereof, those skilled in the art will appreciate that various modifications may be made without departing from the spirit and scope of the invention. The scope of the appended claims is not to be limited to the specific embodiments described.

What is claimed is:

1. A method for treating subcutaneous brown fat and/or subcutaneous beige fat, said method comprising administering an effective amount of a cold solution to a treatment site of a subject, wherein the treatment site is superficial subcutaneous adipose tissue, deep subcutaneous adipose tissue or superficial subcutaneous adipose tissue and deep subcutaneous adipose tissue, and wherein the cold solution comprises liquid water and/or solid ice particles.

2. A method for treating visceral brown fat and/or visceral beige fat, said method comprising administering an effective amount of a cold solution to a treatment site of a subject, wherein the treatment site is visceral adipose tissue, and wherein the cold solution comprises liquid water and/or solid ice particles.

3. The method for treatment of claim 1, wherein the cold solution comprises about 2% to about 70% solid ice particles, and optionally one or more additives.

4. The method for treatment of claim 1, wherein the cold solution comprises about 71% to about 100% solid ice particles, and optionally one or more additives.

5. The method for treatment of claim 1, wherein the cold solution is substantially liquid, and optionally comprises one or more additives.

6. The method for treatment of claim 2, wherein the cold solution comprises about 2% to about 70% solid ice particles, and optionally one or more additives.

7. The method for treatment of claim 2, wherein the cold solution comprises about 71% to about 100% solid ice particles, and optionally one or more additives.

8. The method for treatment of claim 2, wherein the cold solution is substantially liquid, and optionally comprises one or more additives.

9. A method for activating brown fat, said method comprising administering an effective amount of a cold solution to a treatment site of a subject in need thereof, wherein the treatment site is selected from the group consisting of superficial subcutaneous adipose tissue, deep subcutaneous adipose tissue, superficial subcutaneous adipose tissue and deep subcutaneous adipose tissue, visceral adipose tissue, visceral adipose tissue and superficial subcutaneous adipose tissue, visceral adipose tissue and deep subcutaneous adipose tissue, or visceral adipose tissue, superficial subcutaneous adipose tissue and deep subcutaneous adipose tissue, wherein the cold solution comprises liquid water and/or solid ice particles, and optionally one or more additives, and wherein the method results in activation of brown adipocytes, proliferation of brown adipocytes, increased volume of brown adipocytes, cryolipolysis of white adipocytes, conversion of white adipocytes into beige or brown adipocytes, conversion of white precursors into beige adipocytes, conversion of beige precursors into beige adipocytes, and/or conversion of brown precursors into brown adipocytes.

10. The method for treatment of claim 9, wherein the cold solution comprises about 2% to about 70% solid ice particles, and optionally one or more additives.

11. The method for treatment of claim 9, wherein the cold solution comprises about 71% to about 100% solid ice particles, and optionally one or more additives.

12. The method for treatment of claim 9, wherein the cold solution is substantially liquid, and optionally comprises one or more additives.

13. The method for treatment of claim 9, wherein prior to administering an effective amount of the cold solution, imaging the treatment site of the subject to determine the presence of brown adipocytes, beige adipocytes, white adipocytes, and/or precursors of brown, beige and/or white adipocytes.

14. The method for treatment of claim 9, wherein after administering an effective amount of the cold solution, imaging the treatment site, in order to monitor the activation of brown fat.

15. The method for treatment of claim 13, wherein the cold solution is administered in a single treatment, or in a series of treatments.

16. The method for treatment of claim 14, wherein the cold solution is administered in a single treatment, or in a series of treatments.

17. The method for treatment according to claim 9, wherein the cold solution is administered via a device selected from the group consisting of a needle, an expandable needle, a needle comprising more than one tip, a fenestrated needle, a fenestrated cannula, and an implant.

18. A method for increasing metabolic function, said method comprising administering an effective amount of a cold solution to a treatment site of a subject in need thereof, wherein the treatment site is selected from the group consisting of superficial subcutaneous adipose tissue, deep subcutaneous adipose tissue, superficial subcutaneous adipose tissue and deep subcutaneous adipose tissue, visceral adipose tissue, visceral adipose tissue and superficial subcutaneous adipose tissue, visceral adipose tissue and deep subcutaneous adipose tissue, or visceral adipose tissue, superficial subcutaneous adipose tissue and deep subcutaneous adipose tissue, wherein the cold solution comprises liquid water and/or solid ice particles, and optionally one or more additives, and wherein the method results in activation of brown adipocytes, proliferation of brown adipocytes, increased volume of brown adipocytes, cryolipolysis of white adipocytes, conversion of white adipocytes into beige or brown adipocytes, conversion of white precursors into beige adipocytes, conversion of beige precursors into beige adipocytes, and/or conversion of brown precursors into brown adipocytes, thus increasing the metabolic function of the subject.

19. The method for treatment of claim 18, wherein the cold solution comprises about 2% to about 70% solid ice particles, and optionally one or more additives.

20. The method for treatment of claim 18, wherein the cold solution comprises about 71% to about 100% solid ice particles, and optionally one or more additives.

21. The method for treatment of claim 18, wherein the cold solution is substantially liquid, and optionally comprises one or more additives.

22. The method for treatment of claim 18, wherein prior to administering an effective amount of a cold solution, imaging the treatment site of the subject to determine the presence of brown adipocytes, beige adipocytes, white adipocytes, and/or precursors of brown, beige and/or white adipocytes.

23. The method for treatment of claim 18, wherein after administering an effective amount of the cold solution, imaging the treatment site, in order to monitor the activation of brown fat.

24. The method for treatment of claim 22, wherein the cold solution is administered in a single treatment, or in a series of treatments.

25. The method for treatment of claim 23, wherein the cold solution is administered in a single treatment, or in a series of treatments.

26. The method for treatment according to claim 18, wherein the cold solution is administered via a device selected from the group consisting of a needle, an expandable needle, a needle comprising more than one tip, a fenestrated needle, a fenestrated cannula, and an implant.

27. A composition comprising a cold solution, wherein:
the cold solution (1) comprises about 2% to about 70% solid ice particles, (2) comprises about 71% to about 100% solid ice particles, or (3) is substantially liquid;
the composition optionally comprises at least one additive; and
the composition is used to treat brown fat in a subject.

28. A method for activating brown adipocytes in a subject in need thereof, said method comprising:
topically cooling the skin of the subject, at or near a treatment site for activation of brown adipocytes,
administering a β3 receptor agonist to the subject,
imaging the treatment site and surrounding area,
administering a cold solution to the treatment site of the subject via injection to activate brown adipocytes,
wherein the first and second cold solution may be the same or different, and
wherein the first and second cold solutions independently comprise liquid water and/or ice particles, and optionally one or more additives.

29. A method for treating brown adipose tissue in a subject, said method comprising:
imaging one or more potential treatment sites in the subject to determine the presence of brown adipocytes,
selecting one or more treatment sites which contain brown adipocytes, and
administering a cold solution into to the one or more selected treatment sites to treat the brown adipose tissue of the subject,
wherein the cold solution comprises liquid water and/or ice particles, optionally one or more additives, and optionally one or more suitable pharmacological agents.

30. A method for treating brown adipose tissue in a subject, said method comprising:
imaging one or more potential treatment sites in the subject to visualize sympathetic innervation of adipose tissue, and
administering a cold solution into a treatment site with sympathetic innervation, thereby treating brown adipose tissue,
wherein the cold solution comprises liquid water and/or ice particles, optionally one or more additives, and optionally one or more suitable pharmacological agents.

31. A method for treating brown and/or beige adipocytes in a subject, said method comprising:
identifying treatment sites comprising brown and/or beige adipocytes by thermal imaging of the subject,
selecting one or more treatment sites containing brown and/or beige adipocytes,
administering a cold solution to the one or more selected treatment sites to treat the brown and/or beige adipocytes of the subject,
wherein the cold solution comprises liquid water and/or ice particles, optionally one or more additives, and optionally one or more suitable pharmacological agents.

32. A method for treating brown and/or beige adipocytes in a subject, said method comprising:
selecting a treatment site known to contain brown and/or beige adipocytes based upon anatomical location,
administering a cold solution to the one or more selected treatment sites of the subject to treat the brown and/or beige adipocytes of the subject,
wherein the cold solution comprises liquid water and/or ice particles, optionally one or more additives, and optionally one or more suitable pharmacological agents.

33. A method for forming beige adipocytes in a subject, said method comprising:
imaging one or more treatment sites of the subject to determine the presence of white precursors,
administering a cold solution to one or more treatment sites containing white precursors and the surrounding area in order to form beige adipocytes from the white precursors,
wherein the cold solution comprises liquid water and/or ice particles, optionally one or more additives, and optionally one or more suitable pharmacological agents.

34. A method for inducing cryolipolysis of white adipocytes and formation of beige adipocytes in a subject,
imaging one or more treatment sites of the subject to determine the presence of white precursors,
administering a cold solution to one or more treatment sites containing white precursors and the surrounding area in order to form beige adipocytes from the white precursors and to induce cryolipolysis of surrounding white adipocytes,
wherein the cold solution comprises liquid water and/or ice particles, optionally one or more additives, and optionally one or more suitable pharmacological agents.

35. A method for forming beige and/or brown adipocytes in a subject, said method comprising:
imaging one or more treatment sites of the subject to determine the presence of beige and/or brown precursors,
administering a cold solution to one or more treatment sites containing brown and/or beige precursors and the surrounding area in order to form beige and/or brown adipocytes from the beige and/or brown precursors,
wherein the cold solution comprises liquid water and/or ice particles, optionally one or more additives, and optionally one or more suitable pharmacological agents.

36. A method for forming beige and/or brown adipocytes in a subject and inducing cryolipolysis of white adipocytes, said method comprising:
imaging one or more treatment sites of the subject to determine the presence of beige and/or brown precursors,
administering a cold solution to one or more treatment sites containing brown and/or beige precursors and the surrounding area in order to form beige and/or brown adipocytes from the beige and/or brown precursors, and to induce cryolipolysis of surrounding white adipocytes,
wherein the cold solution comprises liquid water and/or ice particles, optionally one or more additives, and optionally one or more suitable pharmacological agents.

37. A method for inducing cryolipolysis of white adipocytes and effecting transdifferentiation from white adipocytes to beige adipocytes, said method comprising:
imaging one or more treatment sites of the subject to determine the presence of beige adipocytes,
administering a cold solution to white adipocytes surrounding the beige adipocytes, thus inducing cryolipolysis of a portion of the white adipocytes while leaving one or more white adipocytes remain, and to effect transdifferentiation from the remaining white adipocytes to beige adipocytes,
wherein the cold solution comprises liquid water and/or ice particles, optionally one or more additives, and optionally one or more suitable pharmacological agents.

38. A method for inducing cryolipolysis of white adipocytes and effecting transdifferentiation from white adipocytes to beige adipocytes, said method comprising:
imaging one or more treatment sites of the subject to determine the presence of beige adipocytes,
administering a first cold solution to white adipocytes surrounding the beige adipocytes, thus inducing cryolipolysis of a portion of the white adipocytes, while leaving one or more white adipocytes remain, and
administering a second cold solution to effect transdifferentiation from the remaining white adipocytes to beige adipocytes,
wherein the first and second cold solution may be the same or different, and
wherein the first and second cold solutions independently comprise liquid water and/or ice particles, and optionally one or more additives.

39. A system for treating brown adipose tissue, the system comprising:
an imaging device,
a delivery device for delivering a cold solution comprising liquid water and/or solid particles to a treatment site of a subject, and
a cold solution supply source configured to supply the cold solution.

40. A system for inducing cryolipolysis of white adipocytes and effecting transdifferentiation from white adipocytes to beige adipocytes, the system comprising:
an imaging device,
a first delivery device for delivering a first cold solution comprising liquid water and/or solid particles to a treatment site of a subject, and optionally a second cold solution comprising liquid water and/or solid particles to a treatment site of the subject,
optionally a second delivery device for delivering a second cold solution comprising liquid water and/or solid particles to a second treatment site of the subject,
wherein the first and second cold solution may be the same or different,
a first cold solution supply source configured to supply the first cold solution and optionally the second cold solution, and
optionally a second cold solution supply source configured to supply the second cold solution.

41. A method for increasing the volume of brown fat in a subject, said method comprising
imaging one or more treatment sites of the subject to determine the presence of brown adipocytes,
administering a cold solution to the one or more treatment sites comprising brown adipocytes to activate and proliferate the brown adipocytes,
and optionally imaging the one or more treatment sites to determine the increase in volume of brown adipocytes,
wherein cold solution comprises liquid water and/or ice particles, and optionally one or more additives.

42. A method for increasing the volume of brown fat in a subject, said method comprising
selecting one or more treatment sites of the subject comprising brown adipocytes, wherein the one or more treatment sites are selected from the adipose tissue between the shoulder blades, in the neck region, in the inter-scapular region, in the supraclavicular area, in the mediastinum area, in the paravertebral area, in the suprarenal area, in the area surrounding the kidneys, and in areas along the gastrointestinal tract,
imaging the one or more treatment sites to determine the amount of brown adipocytes present therein,
administering a cold solution to the one or more treatment sites to activate and proliferate the brown adipocytes present therein,
and optionally imaging the one or more treatment sites to determine the increase in volume of brown adipocytes,
wherein cold solution comprises liquid water and/or ice particles, and optionally one or more additives.

43. The method of claim 41, wherein the cold solution is administered in a single treatment, or as a series of treatments.

44. The method of claim 41, wherein the cold solution is administered via a device selected form the group consisting of a needle, an expandable needle, a needle comprising more than one tip, a fenestrated needle, a fenestrated cannula, and an implant.

45. The method of claim 42, wherein the cold solution is administered in a single treatment, or as a series of treatments.

46. The method of claim 42, wherein the cold solution is administered via a device selected from the group consisting of a needle, an expandable needle, a needle comprising more than one tip, a fenestrated needle, a fenestrated cannula, and an implant.
